SEQUENCE LISTING (53 sequences)

SEQ ID NO: 1 - DNA, 417 bp, Mus musculus
  1A12D11 Heavy chain
atgaactttgtgctcagcttgattttccttgccctcattttaaaaggtgtccagtgtgaagtgcagctggtggagtctgggggaggcttagtgaagcctggagggtccctgaaactctcctgtgcagcctctggattcactttcagtagctatgccatgtcttgggttcgccagactccggagaagaggctggagtgggtcgcaaccattactagtggtggtagttacacctactatacagacagtgtgaagggtcgattcaccatctccagagacaatgccaagaacaccctgtacctgcaaatgagcagtctgaggtctgaggacacggccatgtattactgtgcaagacttatgattacgactggctggtacttcgatgtctggggcgcagggaccacggtcaccgtctcctca

SEQ ID NO: 2 - DNA, 393 bp, Mus musculus
  1A12D11 Light chain
atgaagttgcctgttaggctgttggtgctgatgttctggattcctgcttccagcagtgatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatattccattcacgttcggctcggggacaaagttggaaataaaa

SEQ ID NO: 3 - protein, 139 aa, Mus musculus
  1A12D11 Heavy chain
MNFVLSLIFLALILKGVQCEVQLVESGGGLVKPGGSLKLSCAASGFTFSSYAMSWVRQTPEKRLEWVATITSGGSYTYYTDSVKGRFTISRDNAKNTLYLQMSSLRSEDTAMYYCARLMITTGWYFDVWGAGTTVTVSS

SEQ ID NO: 4 - protein, 131 aa, Mus musculus
  1A12D11 Light chain
MKLPVRLLVLMFWIPASSSDVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHIPFTFGSGTKLEIK

SEQ ID NO: 5 - protein, 5 aa, Mus musculus
  1A12D11 Heavy chain CDR1
SYAMS

SEQ ID NO: 6 - protein, 17 aa, Mus musculus
  1A12D11 Heavy chain CDR2
TITSGGSYTYYTDSVKG

SEQ ID NO: 7 - protein, 11 aa, Mus musculus
  1A12D11 Heavy chain CDR3
LMITTGWYFDV

SEQ ID NO: 8 - protein, 16 aa, Mus musculus
  1A12D11 Light chain CDR1
RSSQSIVHSNGNTYLE

SEQ ID NO: 9 - protein, 7 aa, Mus musculus
  1A12D11 Light chain CDR2
KVSNRFS

SEQ ID NO: 10 - protein, 9 aa, Mus musculus
  1A12D11 Light chain CDR3
FQGSHIPFT

SEQ ID NO: 11 - DNA, 402 bp, Mus musculus
  3G6D9 Heavy chain
atgtacttgggactgaactgtgtattcatagtttttctcttaaaaggtgtccagagtgaagtgaagcttgaggagtctggaggaggcttggtgcaacctggaggatccatgaaactctcctgtgttgcctctggattcactttcagtaactactggatgaactgggtccgccagtctccagagaaggggcttgagtgggttgctgaaattagattgaaatctaataattatgcaacacattatgcggagtctgtgaaagggaggttcaccatctcaagagatgattccaaaagtagtgtctacctgcaaatgaacaacttaagaactgaagacactggcatttattactgtacccttacccttgactactggggccaaggcaccactctcacagtctcctca

SEQ ID NO: 12 - DNA, 399 bp, Mus musculus
  3G6D9 Light chain
atggattcacaggcccaggttcttatgttactgctgctatgggtatctggtacctgtggggacattgtgatgtcacagtctccatcctccctagctgtgtcagttggagaggaggttactatgagctgcaagtccagtcagagccttttatacagtaccaatcaaaagaactacttggcctggtaccagcagaaaccagggcagtctcctaaactgctgatttactgggcatccactagggagtctggggtccctgatcgcttcacaggcagtggatctgggacagatttcactctcaccatcagcagtgtgaaggctgaagacctggcagtttattactgtctgcaatattatagctatccgtacacgttcggaggggggaccaagctggaaataaaa

SEQ ID NO: 13 - protein, 134 aa, Mus musculus
  3G6D9 Heavy chain
MYLGLNCVFIVFLLKGVQSEVKLEESGGGLVQPGGSMKLSCVASGFTFSNYWMNWVRQSPEKGLEWVAEIRLKSNNYATHYAESVKGRFTISRDDSKSSVYLQMNNLRTEDTGIYYCTLTLDYWGQGTTLTVSS

SEQ ID NO: 14 - protein, 133 aa, Mus musculus
  3G6D9 Light chain
MDSQAQVLMLLLLWVSGTCGDIVMSQSPSSLAVSVGEEVTMSCKSSQSLLYSTNQKNYLAWYQQKPGQSPKLLIYWASTRESGVPDRFTGSGSGTDFTLTISSVKAEDLAVYYCLQYYSYPYTFGGGTKLEIK

SEQ ID NO: 15 - protein, 5 aa, Mus musculus
  3G6D9 Heavy chain CDR1
NYWMN

SEQ ID NO: 16 - protein, 19 aa, Mus musculus
  3G6D9 Heavy chain CDR2
EIRLKSNNYATHYAESVKG

SEQ ID NO: 17 - protein, 4 aa, Mus musculus
  3G6D9 Heavy chain CDR3
TLDY

SEQ ID NO: 18 - protein, 17 aa, Mus musculus
  3G6D9 Light chain CDR1
KSSQSLLYSTNQKNYLA

SEQ ID NO: 19 - protein, 7 aa, Mus musculus
  3G6D9 Light chain CDR2
WASTRES

SEQ ID NO: 20 - protein, 9 aa, Mus musculus
  3G6D9 Light chain CDR3
LQYYSYPYT

SEQ ID NO: 21 - DNA, 393 bp, Mus musculus
  5D11B9 Heavy chain
atgaaatgcagctgggtcatcttcttcctgatggcagtggttacaggggtcaattcagaggttcagctgcagcagtctggggctgagcttgtgaggccaggggccttagtcaagttgtcctgcaaagcttctggcttcaacattaaagactactatatacactgggtgaagcagaggcctgaacagggcctggagtggattggatggattaatcctgagaatggtaatactatgtatgacccgaagttccagggcaaggccagtataacagcagacacatcctccaacacagcctacctgcagctcagcggcctgacatctgaggacactgccgtctattactgtgctagagatgtctactggggccaagggactctggtcactgtctctgca

SEQ ID NO: 22 - DNA, 393 bp, Mus musculus
  3G6D9 Light chain
atgaagttgcctgttaggctgttggtgctgatgttctggattcctgcttccagcagtgatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttcctctcacgttcggtgctgggaccaagctggagctgaaa

SEQ ID NO: 23 - protein, 131 aa, Mus musculus
  5D11B9 Heavy chain
MKCSWVIFFLMAVVTGVNSEVQLQQSGAELVRPGALVKLSCKASGFNIKDYYIHWVKQRPEQGLEWIGWINPENGNTMYDPKFQGKASITADTSSNTAYLQLSGLTSEDTAVYYCARDVYWGQGTLVTVSA

SEQ ID NO: 24 - protein, 131 aa, Mus musculus
  5D11B9 Light chain
MKLPVRLLVLMFWIPASSSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPLTFGAGTKLELK

SEQ ID NO: 25 - protein, 5 aa, Mus musculus
  5D11B9 Heavy chain CDR1
DYYIH

SEQ ID NO: 26 - protein, 17 aa, Mus musculus
  5D11B9 Heavy chain CDR2
WINPENGNTMYDPKFQG

SEQ ID NO: 27 - protein, 4 aa, Mus musculus
  5D11B9 Heavy chain CDR3 3
DVYW

SEQ ID NO: 28 - protein, 16 aa, Mus musculus
  5D11B9 or 9H2G7 Light chain CDR1
RSSQSLVHSNGNTYLH

SEQ ID NO: 29 - protein, 7 aa, Mus musculus
  5D11B9 or 9H2G7 Light chain CDR2
KVSNRFS

SEQ ID NO: 30 - protein, 9 aa, Mus musculus
  5D11B9 or 9H2G7 Light chain CDR3
SQSTHVPLT

SEQ ID NO: 31 - DNA, 393 bp, Mus musculus
  9H2G7 Heavy chain
atgggatggagctggatctttctctttctcctgtcaggaactgcaggtgtcctctctgaggtccagctgcaacagtctggacctgagctggtgaagcctggggcttcagtgaagatatcctgcaaggcttctggttactcattcactggctactacatgcactgggtgaagcaaagccatgtaaagagccttgagtggattggacatattaatccttacaatggtgctactagctacaaccagaatttcaaggacaaggccagcttgactgtagataagtcctccagcacagcctacatggagctccgcagcctgacatctgaggactctgcagtctattactgtgcaagagatacttactggggccaagggactctggtcactgtctctgca

SEQ ID NO: 32 - DNA, 393 bp, Mus musculus
  3G6D9 Light chain
atgaagttgcctgttaggctgttggtgctgatgttctggattcctgcttccagcagtgatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgctcacgttcggtgctgggaccaagctggagctgaaa

SEQ ID NO: 33 - protein, 131 aa, Mus musculus
  9H2G7 Heavy chain
MGWSWIFLFLLSGTAGVLSEVQLQQSGPELVKPGASVKISCKASGYSFTGYYMHWVKQSHVKSLEWIGHINPYNGATSYNQNFKDKASLTVDKSSSTAYMELRSLTSEDSAVYYCARDTYWGQGTLVTVSA

SEQ ID NO: 34 - protein, 131 aa, Mus musculus; identical to SEQ ID NO: 24
  9H2G7 Light chain

SEQ ID NO: 35 - protein, 5 aa, Mus musculus
  9H2G7 Heavy chain CDR1
GYYMH

SEQ ID NO: 36 - protein, 17 aa, Mus musculus
  9H2G7 Heavy chain CDR2
HINPYNGATSYNQNFKD

SEQ ID NO: 37 - protein, 4 aa, Mus musculus
  9H2G7 Heavy chain CDR3 3
DTYW

SEQ ID NO: 38 - protein, 14 aa, Homo sapiens
  Immunogen IFITM-2
KEEQEVAMLGVPHN

SEQ ID NO: 39 - protein, 14 aa, Homo sapiens
  Immunogen IFITM-3
KEEHEVAVLGAPHN

SEQ ID NO: 40 - protein, 56 aa, Homo sapiens
  hIFITM-2 (1-56 aa)
MNHIVQTFSPVNSGQPPNYEMLKEEQEVAMLGVPHNPAPPMSTVIHIRSETSVPDH

SEQ ID NO: 41 - protein, 57 aa, Homo sapiens
  hIFITM-3 (1-57 aa)
MNHTVQTFFSPVNSGQPPNYEMLKEEHEVAVLGAPHNPAPPTSTVIHIRSETSVPDH

SEQ ID NO: 42 - DNA, 20 bp, Unknown
  Viruses
aggttgatcacaggcagact

SEQ ID NO: 43 - DNA, 18 bp, Unknown
  Viruses
gctgactgagggaaggac

SEQ ID NO: 44 - DNA, 18 bp, Mus musculus
  Primer For GAPDHC
cctttcattgagctccat

SEQ ID NO: 45 - DNA, 16 bp, Mus musculus
  Primer Rev GAPDH
cgtacatgggagcgtc

SEQ ID NO: 46 - DNA, 20 bp, Homo sapiens
  Primer for Human IFITM2
acggaactactggggaaagg

SEQ ID NO: 47 - DNA, 20 bp, Homo sapiens
  primer rev Human IFITM2
cacttcctgctcctccttga

SEQ ID NO: 48 - DNA, 21 bp, Homo sapiens
  primer for Human b actin
tgacccagatcatgtttgaga

SEQ ID NO: 49 - DNA, 20 bp, Homo sapiens
  primer rev Human b actin
agggcatacccctcgtagat

SEQ ID NO: 50 - RNA, 21 nt, Homo sapiens
  IFITM2 N-Terminal 5′ siRNA
aaccuucucuccugucaacag

SEQ ID NO: 51 - RNA, 21 nt, Homo sapiens
  IFITM2 N-Terminal 3′ siRNA
uuggaagagaggacaguuguc

SEQ ID NO: 52 - RNA, 21 nt, Homo sapiens
  IFITM2 C-Terminal 5′ siRNA
uuggucguccaggcccagcgu

SEQ ID NO: 53 - RNA, 21 nt, Homo sapiens
  IFITM2 C-Terminal 3′ siRNA
aaccagcagguccgggucgca

(19) United States
(12) Patent Application Publication
(10) Pub. No.: US 2025/0320308 A1
(43) Pub. Date: Oct. 16, 2025

(54) MONOCLONAL ANTIBODIES FOR THE TREATMENT OF VIRAL INFECTIONS

(71) Applicant: FIBROSYS S.R.L., Baronissi (SA) (IT)

(72) Inventors: Liberato MARZULLO, Baronissi (SA) (IT); Maria Caterina TURCO, Avellino (AV) (IT); Alessandra ROSATI, Baronissi (SA) (IT); Margot DE MARCO, Salerno (SA) (IT); Anna BASILE, Bracigliano (SA) (IT); Gianluigi FRANCI, Napoli (NA) (IT); Carla ZANNELLA, Mugnano di Napoli (NA) (IT)

(21) Appl. No.: 18/280,540

(22) PCT Filed: Mar. 11, 2022
(86) PCT No.: PCT/EP2022/056343

§ 371 Date: Sep. 6, 2023

(30) Foreign Application Priority Data
Mar. 12, 2021 (IT) 102021000005909

(51) Int. Cl. C07K 16/28; A61K 45/06; A61P 31/14; A61P 31/22

(52) U.S. Cl. CPC C07K 16/2896; A61K 45/06; A61P 31/14; A61P 31/22; C07K 2317/24; C07K 2317/55; C07K 2317/565; C07K 2317/76

(57) ABSTRACT

The present invention provides for monoclonal anti-IFITM2 and anti-IFITM3 antibodies or a fragment thereof, pharmaceutical compositions comprising said antibodies and its use in the treatment of viral infections, preferably of SARS-COV-2 infection in host human cells.

### BACKGROUND OF THE INVENTION

In December 2019, an outbreak of pneumonia cases of unknown etiology was reported in Wuhan (Hubei Province, China), from the Chinese health authorities. Early, in 2020 the Chinese Centre for Disease Control and Prevention (CDC) recognized a new coronavirus as the responsible agent of the epidemic. The new Coronavirus was associated with the severe acute respiratory syndrome (SARS) coronaviruses (SARS [symbol] CoVs), and named SARS [symbol] COV [symbol] 2 by the Coronavirus Study Group (CSG) of the International Committee on Taxonomy of Viruses. SARS-COV-2 turned out to be very contagious and on Mar. 11, 2020 the World Health Organization (WHO) declared COVID [symbol] 19, the SARS-COV-2 related disease, as a global pandemic. Thereafter the virus spread to more than 200 countries, with severe public health and economic consequences. In particular, some European Countries such as Italy, Spain and the UK became global hotspots in March 2020 immediately after Asia outbreak. From mid-April the infections' focus shifted to the US continuing to remain consistently high. Then Latin America surged as the center of the epidemic. Recently, the increasing numbers of SARS-COV-2 positive people in India and a second wave in Europe established that COVID-19 is still a global pandemic problem.

Coronaviruses are a large family of enveloped single-stranded RNA viruses (+ssRNA) that can be isolated in different animal species. Coronaviruses that infect mammals (except pigs) belong mainly to two genetic and serologic groups: the Alpha-and Betacoronavirus genera. The evolutionary analysis suggested that the lineage from which SARS-COV-2, a Betacoronavirus, emerged has been present in bats for several decades.

SARS-COV-2 has been identified as the seventh coronavirus known to infect humans; previous six were: HCoV-299E, HCoV-NL63, HCoV-HKU, HCoV-OC43, MERS-COV and SARS-COV. Interestingly, these two latter viruses have probably originated from bats and then moving into other mammalian hosts before jumping to humans. SARS-COV mammalian intermediary was identified in the Himalayan palm civet, while for the MERS-COV was the dromedary camel. For the SARS-COV-2 enigma some have hypothesized that the pangolin could be the missing link. The genetic makeup of SARS-COV-2 is composed of 13-15 open reading frames (ORFs) containing ˜30,000 nucleotides. The six functional open reading frames (ORFs) are arranged in order from 5′ to 3′: replicase (ORF1a/ORF1b), spike(S), envelope (E), membrane (M) and nucleocapsid (N). Of the four structural genes, SARS-COV-2 shares more than 90% amino acid identity with SARS-COV except for the S gene, which diverges. The replicase gene covers two thirds of the 5′ genome, and encodes a large polyprotein (pp1ab), which is proteolytically cleaved into 16 non-structural proteins that are involved in transcription and virus replication (Hu et al., Nat Rev Microbiol; 2020).

Infection starts when the Spike protein that protrudes on the virion surface of SARS-CoV-2 binds to ACE-2 human protein that instead is present on plasma membranes of many important human cells, including type II alveolar cells in the lungs. When the viral key (Spike) opens the door (cell plasma membrane) through the lock (ACE-2), the virus is able to infiltrate the cell and replicate. When into the cells, viruses deviate host cell metabolism and forces it to create copies of its biological code. Experts are still trying to understand how this novel coronavirus is able to attack the body and to decode mechanisms that hint immune system overreaction with deadly consequences. One of the possible mechanisms through which SARS-COV-2 could efficiently infect a broad range of host human cells is hijacking IFITM human proteins.

IFITM genes are a subfamily of the larger family of Dispanin, characterized by a common two Trans-Membrane (2TM) structure that in vertebrates can be classified into four subfamilies (A-D). Dispanins are essentially present in metazoan and surprisingly in several bacterial phyla. The phylogenetic studies evidenced high sequence similarities and conserved sequence motifs among eukaryotes and bacteria thus suggesting functional relationships and most probably a bacterial origin of the proteins, later introduced in eukaryotes by horizontal gene transfer. The DSPA subfamily encompasses six human genes, DSPA1, DSPA2a-d and DSPA3, and among them DSPA2a, 2b and 2c correspond to genes encoding IFITM-1, -2 and -3. IFITM proteins were discovered more than 20 years ago during a screening for proteins induced by interferon (Friedman et al., 1984), but only in 2009 it was shown their activity as anti-viral restriction factors able to confer basal and IFN-induced resistance to Influenza A virus and to flaviviruses (Dengue, West Nile) (Brass et al., 2009). Among the other members of the family IFITM1, IFITM2 and IFITM3 were also termed immune-related IFITMs, because of their ability to inhibit the viral entry and host/virus membrane fusion, even though they have been associated, along with the other members of the protein family, to several distinct biological functions like germ cell specification, osteoblast function and bone mineralization (IFITM5), immune functions, cell cycle control and apoptosis (Yànez et al., 2020).

Based on sequence similarity and putative functions, human IFITM1, IFITM2 and IFITM3 can be grouped in a clade where IFITM1 modestly diverges from the highly homologous IFITM2 and IFITM3 (about 90% identity). Differences of their respective primary structures also affect their subcellular localization and trafficking. In fact, they are mainly localized in the endo-lysosomal compartment, but the subcellular distribution varies with their expression level and cell or tissue type, and their vesicular trafficking with the plasma membrane can be clathrin- (IFITM2 and IFITM3 N-terminal domain contains the conserved YXXϕ motif binding the clathrin adaptor AP-2 protein) or caveolin- (IFITM1) dependent.

The membrane topology of the IFITM proteins is still under debate due to conflicting pieces of evidence about the orientation of the N- and C-termini, while the presence of a common intracellular loop (CIL) and two trans-/intra-membrane domains are well conserved and widely accepted. Structural hypotheses based on experimental evidence are compatible with three alternative modelling: an intramembrane topology having both N- and C-termini pointing inward into the cytosol, and two other possible structures having a luminal C-terminus, and the N-terminus (NTD) alternatively exposed to the cytosol (Type II TM topology), or to the lumen (Type III TM topology). Functional studies demonstrated that biological activities of IFITM proteins strongly depend on post-translational modifications of CIL and NTD domains of the proteins (Bailey et al., 2014). Hence, it is self-evident that molecular tools able to distinguish their topology are of outstanding importance to dissect molecular mechanisms regulating their biological activities, and even more when highly homologous proteins like IFITM2 and IFITM3 could erroneously let infer a functional redundancy.

It has also been reported that IFITM proteins inhibit human coronaviruses including SARS-COV-1 and SARS-COV-2, as well as MERS-COV (Huang et al., 2011). As reported by Prelli Bozzo et al. 2020 (manuscript available on biorxiv at: bioRxiv 2020.08.18.255935; doi: https://doi.org/10.1101/2020.08.18.255935), most of the results reporting the inhibition of human coronavirus were obtained using Spike containing viral pseudo-particles and cell lines overexpressing the IFITM proteins and, frequently, also the viral ACE2 receptor.

Prelli Bozzo et al., in contrast, showed that endogenous IFITM proteins were essential for efficient infection and replication of genuine SARS-COV-2 in various types of human cells, thus in part explaining the rapid spread of this pandemic viral pathogen. In particular, they showed that IFITM proteins are entry cofactors of SARS-COV-2 in a way that mimicking peptides and/or commercially available antibodies inhibited SARS-COV-2 infection of human lung, heart and gut cells. The results disclosed in Prelli Bozzo et al. therefore report contrasting pieces of evidence, due to the different condition of the experiments made.

Considering the wide spread of SARS-COV-2 infection all over the word, the high mortality observed and that, at the moment, conventional treatments for COVID with anti-inflammatory and/or anti-viral molecules pose numerous drawbacks linked to side effects and are not, at present, definitive means of treating such pathology, there is therefore an evident need for a new and improved therapeutic treatment which has the advantage of being highly specific and having few or no side effects, as compared with the conventional, commonly known therapies used for the treatment of viral infections.

### Definitions

Unless otherwise defined, all terms of art, notations and other scientific terminology used herein areintended to have the meanings commonly understood by those persons skill in the art to which this disclosure pertains. In some cases, terms with commonly understood meanings are defined herein for clarity and/or for ready reference; thus, the inclusion of such definitions herein should not be construed to represent a substantial difference over what is generally understood in the art.

The term “antibody” as used herein includes “fragments” or “derivatives”, which have at least one antigen binding site of the antibody and/or show the same biological activity.

An antibody preferably comprises at least one heavy immunoglobulin chain and at least one light immunoglobulin chain. An immunoglobulin chain comprises a variable domain and optionally a constant domain. A variable domain may comprise complementarity determining regions (CDRs), e.g. a CDR1, CDR2 and/or CDR3region, and framework regions.

The term “humanized antibody” refers to an antibody of human origin, whose hypervariable region has been replaced by the homologous region of non-human monoclonal antibodies.

The term “chimeric antibody” refers to an antibody containing portions derived from different antibodies.

The term “recombinant antibody” refers to an antibody obtained using recombinant DNA methods.

The term “scFv fragment” (single chain variable fragment) refers to immunoglobulin fragments only capable of binding with the antigen concerned. ScFv fragments can also be synthetized into dimers (diabodies), trimers (triabodies) and tetramers (tetrabodies) using peptide linkers.

The terms “Fab fragment” (antigen-binding fragment) and “F(ab′)₂ fragment” refer to immunoglobulin fragments consisting of a light chain linked to the Fc fragment of the adjacent heavy chain, and such fragments are monovalent antibodies. When the Fab portions are in pairs, the fragment is called F(ab′)₂.

The term “hybridoma” refers to a cell producing monoclonal antibodies.

The term “monospecific antibodies” refers to antibodies that all have affinity for the same antigen.

The term “multispecific antibodies” refers to antibodies that have affinity for several antigens.

The term “bispecific antibody” refers to an antibody that has affinity for two different antigens.

### DESCRIPTION OF THE FIGURES

FIG. 1. Discloses the sequence alignment and the antigen sequences. In particular Immunogen IFITM2 has SEQ ID NO: 40, Immunogen IFITM3 has SEQ ID NO: 41, Ag-Nh-IFI12 has SEQ ID NO: 38 and Ag-Nh-IFI13 has SEQ ID NO: 39.

FIG. 2. Discloses the hybridoma clones and murine IgG isotypes.

FIG. 3. Western blot analysis of hybridoma clones' supernatants antigen binding test.

FIGS. 4A and B. Antigen binding test for monoclonal antibodies by ELISA assay.

FIGS. 5A and B. Results from mAb 5D11B9 titration on binding Vero E6 cells by FACS analysis.

FIGS. 6A and B. Results from mAb 3G6D9 titration on binding Vero E6 cells by FACS analysis.

FIGS. 7A and B. Results from mAb 1A12D11 titration on binding Vero E6 cells by FACS analysis.

FIGS. 8A and B. Results from peptide competition assay on Vero E6 cells for mAb 5D11B9 by FACS analysis.

FIGS. 9A and B. Results from peptide competition assay on Vero E6 cells for mAb 3G6D9 by FACS analysis.

FIG. 10. Inhibition of SARS-COV-2 infection on Vero E6 cells by mAb 3G6D9 and by mAb 5D11B9 (plaque forming assay).

FIG. 11. Inhibition of SARS-COV-2 infection on Vero E6 cells by mAb 3G6D9 (RT-PCR analysis on SARS-COV-2 Spike gene).

FIGS. 12 (A, B, C and D). Binding to human IFITMs recombinant proteins and peptides: comparison with commercially available Abs.

FIG. 13. Sars-COV-2 infection in Vero E6 cells (PFU assay): comparison with commercially available Abs.

FIGS. 14A and B. A recombinant 5D11B9 mAb showed similar activity in an antigen binding ELISA test and similar efficacy in inhibiting Sars-COV-2 infection by plaque assay.

FIGS. 15A and B. 5D11B9 mAb showed also efficacy in inhibiting HSVs and OC43-induced plaque formation.

FIGS. 16A and B. Binding of 5D11B9 mAb on Human Lung Adenocarcinoma Calu-3 cells is reduced by IFITM2 specific silencing (siRNAs).

FIG. 17. 5D11B9 mAb does not affect the ACE-2 activity in Vero E6 and in Calu-3 cells.

FIGS. 18A and B. 5D11B9 mAb increases recombinant Sars-COV-2 accumulation on Vero E6 cell surface.

FIG. 19. 5D11B9 mAb prevents Sars-COV-2 Spike protein entry in human Calu-3 cells.

### DISCLOSURE OF THE INVENTION

In the present invention, we have identified new anti-IFITM2 and anti-IFITM3 monoclonal recombinant antibodies, that were particularly effective in binding IFITM2 and IFITM3 proteins on human host cell surface and to inhibit the entry of viruses in host human cells, preferably the entry of SARS-COV-2. Here, it has been described a new receptorial or co-receptorial role of IFITM proteins to date not yet fully elucidated.

In particular, inventors have developed monoclonal antibodies capable of binding IFITM2 and IFITM3 proteins when exposed on the cell surface; data collected so far have in fact highlighted the inhibitory action on the viral infection mediated by the antibodies produced by the inventors. The use of monoclonal antibodies capable of inhibiting the binding with IFITM2/3 membrane proteins may constitute an effective extension of the therapeutic and prophylactic antiviral tools against SARS-COV-2, and potentially against other viruses, with particular attention to cases of contraindication for vaccination prophylaxis and in association with current reference therapies.

The invention disclosed here refers to monoclonal antibodies constructed to recognize N-terminal domain of IFITM2 and IFITM3 antibodies and used to inhibit the virus entry into host human cells, preferably of the Sars-COV-2 virus.

Inventors selected suitable antigen candidates for monoclonal antibodies production and assumed the hypothesis of the type III TM topology for IFITM2 and IFITM3, characterized by the extracellular exposure of both N- and C-termini (NTD and CTD), respectively linked to the anchoring part of the protein made by two antiparallel transmembrane domains (TM1 and TM2) and a short Cytosolic Loop (CIL).

To this aim, we analyzed the N-terminal domain sequences of IFITM2 and IFITM3 and selected short sequence stretches encompassing the highest number of amino acid substitutions between the two sequences to synthesize oligopeptides for mice immunization.

After the screening of hybridomas and clone purification we selected 4 clones namely: 5D11B9, 9H2G7, 1A12D11 and 3G6D9, from which we isolated and characterized monoclonal antibodies able to recognize the N-terminal domain of

IFITM2 and IFITM3. Said antibodies are surprisingly able to bind human host cell surface and to inhibit Sars-COV-2 entry into host human cells.

The first embodiment of the present invention is therefore an antibody or a fragment thereof which binds the N-terminal domain of IFITM2 or IFITM3 obtained by immunizing mice with a peptide consisting of an amino acid sequence as in SEQ ID NO: 38 or SEQ ID NO: 39.

A preferred embodiment of the present invention is an antibody or a fragment thereof comprising a combination of a heavy chain or at least the heavy chain variable domain and a light chain or at least the light chain variable domain thereof, selected from the group of combinations consisting of:
a) a heavy chain amino acid sequence as in SEQ ID NO. 3 or at least the variable domain thereof or an amino acid sequence having at least 80% identity with the amino acid sequence in SEQ ID N. 3 or at least the variable domain thereof, and a light chain amino acid sequence as in SEQ ID NO. 4 or at least the variable domain thereof or an amino acid sequence having at least 80% identity with the amino acid sequence in SEQ ID N. 4 or at least the variable domain thereof;
b) a heavy chain amino acid sequence as in SEQ ID NO. 13 or at least the variable domain thereof or an amino acid sequence having at least 80% identity with the amino acid sequence in SEQ ID N: 13 or at least the variable domain thereof, and a light chain amino acid sequence as in SEQ ID NO: 14 or at least the variable domain thereof or an amino acid sequence having at least 80% identity with the amino acid sequence in SEQ ID NO. 14 or at least the variable domain thereof;
c) a heavy chain amino acid sequence as in SEQ ID NO: 23 or at least the variable domain thereof or an amino acid sequence having at least 80% identity with the amino acid sequence as in SEQ ID NO. 23 or at least the variable domain thereof, and a light chain amino acid sequence as in SEQ ID NO: 24 or at least the variable domain thereof or an amino acid sequence having at least 80% identity with the amino acid sequence as in SEQ ID. NO. 24 or at least the variable domain thereof;
d) a heavy chain amino acid sequence as in SEQ ID NO: 33 or at least the variable domain thereof or an amino acid sequence having at least 80% identity with the amino acid sequence as in SEQ ID NO. 33 or at least the variable domain thereof, and a light chain amino acid sequence as in SEQ ID NO: 34 or at least the variable domain thereof or an amino acid sequence having at least 80% identity with the amino acid sequence as in SEQ ID NO. 34 or at least the variable domain thereof. A further preferred embodiment is an antibody or a fragment thereof comprising a combination of a heavy chain or at least the heavy chain variable domain and a light chain or at least the light chain variable domain thereof selected from the group of combinations consisting of:
a) a heavy chain amino acid sequence as in SEQ ID NO. 3 or at least the variable domain thereof or an amino acid sequence having at least 90% identity, preferably at least 95% identity, with the amino acid sequence in SEQ ID N. 3 or at least the variable domain thereof, and a light chain amino acid sequence as in SEQ ID NO. 4 or at least the variable domain thereof or an amino acid sequence having at least 90% identity, preferably at least 95% identity, with the amino acid sequence in SEQ ID N. 4 or at least the variable domain thereof;
b) a heavy chain amino acid sequence as in SEQ ID NO. 13 or at least the variable domain thereof or an amino acid sequence having at least 90% identity, preferably at least 95% identity, with the amino acid sequence in SEQ ID N: 13 or at least the variable domain thereof, and a light chain amino acid sequence as in SEQ ID NO: 14 or at least the variable domain thereof or an amino acid sequence having at least 90% identity, preferably at least 95% identity, with the amino acid sequence in SEQ ID NO. 14 or at least the variable domain thereof;
c) a heavy chain amino acid sequence as in SEQ ID NO: 23 or at least the variable domain thereof or an amino acid sequence having at least 90% identity, preferably at least 95% identity, with the amino acid sequence as in SEQ ID NO. 23 or at least the variable domain thereof, and a light chain amino acid sequence as in SEQ ID NO: 24 or at least the variable domain thereof or an amino acid sequence having at least 90% identity, preferably at least 95% identity, with the amino acid sequence as in SEQ ID. NO. 24 or at least the variable domain thereof;
d) a heavy chain amino acid sequence as in SEQ ID NO: 33 or at least the variable domain thereof or an amino acid sequence having at least 90% identity, preferably at least 95% identity, with the amino acid sequence as in SEQ ID NO. 33 or at least the variable domain thereof, and a light chain amino acid sequence as in SEQ ID NO: 34 or at least the variable domain thereof or an amino acid sequence having at least 90% identity, preferably at least 95% identity, with the amino acid sequence as in SEQ ID NO. 34 or at least the variable domain thereof.

As used herein, “sequence identity” between two polypeptide/amino acid sequences, indicates the percentage of amino acids that are identical between the sequences, preferably over the entire length of the amino acid sequences as in SEQ ID NO: 3 and SEQ ID NO: 4, as in SEQ ID NO: 13 and SEQ ID NO: 14, as in SEQ ID NO: 23 and SEQ ID NO: 24 and as in SEQ ID NO: 33 and SEQ ID NO: 34.

Preferred polypeptide/amino acid sequences of the invention have a sequence identity of at least 85%, more preferably 90%, even more preferably 93%, 94%, 95%, 96%, 97%, 98% or 99%.

In a preferred embodiment the antibody or a fragment thereof of the present invention is the antibody wherein the heavy chain amino acid sequence is SEQ ID NO. 3 and the light chain amino acid sequence is SEQ ID NO 4.

According to a preferred embodiment, wherein the combination of a heavy chain amino acid sequence or at least the heavy chain variable domain thereof and of a light chain amino acid sequence or at least the light chain variable domain thereof is that at point a) the heavy chain amino acid sequence or at least the variable domain thereof, comprises the CDRs regions having the following amino acid composition: H-CDR1 comprises the amino acids SYAMS (SEQ ID N. 5), H-CDR2 comprises the amino acids TITSGGSYTYYTDSVKG (SEQ ID N. 6), H-CDR3 comprises the amino acids LMITTGWYFDV (SEQ ID N. 7) and the light chain amino acid sequence or at least the variable domain thereof comprises the CDRs regions having the following amino acid composition: L-CDR1 comprises the amino acids RSSQSIVHSNGNTYLE (SEQ ID N. 8), L-CDR2 comprises the amino acids KVSNRFS (SEQ ID N. 9) and L-CDR3 comprises the amino acids FQGSHIPFT (SEQ ID N. 10).

In a preferred embodiment the antibody or the fragment thereof of the present invention is the antibody wherein the heavy chain amino acid sequence is SEQ ID NO. 13 and the light chain amino acid sequence is SEQ ID NO 14.

According to a preferred embodiment, wherein the combination of a heavy chain amino acid sequence or at least the heavy chain variable domain thereof and of a light chain amino acid sequence or at least the light chain variable domain thereof is that at point b) the heavy chain amino acid sequence or at least the variable domain thereof comprises the CDRs regions having the following amino acid composition: H-CDR1 comprises the amino acids NYWMN (SEQ ID N. 15), H-CDR2 comprises the amino acids EIRLKSNNYATHYAESVKG (SEQ ID N. 16), H-CDR3 comprises the amino acids TLDY (SEQ ID N. 17) and the light chain amino acid sequence or at least the variable domain thereof comprises the CDRs regions having the following amino acid composition: L-CDR1 comprises the amino acids KSSQSLLYSTNQKNYLA (SEQ ID N. 18), L-CDR2 comprises the amino acids WASTRES (SEQ ID N. 19) and L-CDR3 comprises the amino acids LQYYSYPYT (SEQ ID N. 20).

In a preferred embodiment the antibody or the fragment thereof of the present invention is the antibody wherein the heavy chain amino acid sequence is SEQ ID NO. 23 and the light chain amino acid sequence is SEQ ID NO 24.

According to a preferred embodiment, wherein the combination of a heavy chain amino acid sequence or at least the heavy chain variable domain thereof and of a light chain amino acid sequence or at least the light chain variable domain thereof is that at point c), the heavy chain amino acid sequence or at least the variable domain thereof comprises the CDRs regions having the following amino acid composition: H-CDR1 comprises the amino acids DYYIH (SEQ ID N. 25), H-CDR2 comprises the amino acids WINPENGNTMYDPKFQG (SEQ ID N. 26), H-CDR3 comprises the amino acids DVYW (SEQ ID N. 27) and the light chain amino acid sequence or at least the variable domain thereof comprises the CDRs regions having the following amino acid composition: L-CDR1 comprises the amino acids RSSQSLVHSNGNTYLH (SEQ ID N. 28), L-CDR2 comprises the amino acids KVSNRFS (SEQ ID N. 29) and L-CDR3 comprises the amino acids SQSTHVPLT (SEQ ID N. 30).

In a preferred embodiment the antibody or the fragment thereof of the present invention is the antibody wherein the heavy chain amino acid sequence is SEQ ID NO. 33 and the light chain amino acid sequence is SEQ ID NO 34.

According to a preferred embodiment, wherein the combination of a heavy chain amino acid sequence or at least the heavy chain variable domain thereof and of a light chain amino acid sequence or at least the light chain variable domain thereof is that at point d), the heavy chain amino acid sequence or at least the variable domain thereof comprises the CDRs regions having the following amino acid composition: H-CDR1 comprises the amino acids GYYMH (SEQ ID N. 35), H-CDR2 comprises the amino acids HINPYNGATSYNONFKD (SEQ ID N. 36), H-CDR3 comprises the amino acids DTYW (SEQ ID N. 37) and the light chain amino acid sequence or at least the variable domain thereof, comprises the CDRs regions having the following amino acid composition: L-CDR1 comprises the amino acids RSSQSLVHSNGNTYLH (SEQ ID N. 28), L-CDR2 comprises the amino acids KVSNRFS (SEQ ID N. 29) and L-CDR3 comprises the amino acids SQSTHVPLT (SEQ ID N. 30).

According to a preferred embodiment, the antibody or a fragment thereof according to the present invention, comprises a combination of a heavy chain or at least the heavy chain variable domain and of a light chain or at least the light chain variable domain thereof selected from the group of combination consisting of:
a) a heavy chain nucleotide sequence as in SEQ ID NO: 1 or at least the variable domain thereof or a nucleotide sequence having a sequence identity of at least 80% thereof, and a light chain nucleotide sequence as in SEQ ID NO: 2 or at least the variable domain thereof or a nucleotide sequence having a sequence identity of at least 80% thereof;
b) a heavy chain nucleotide sequence as in SEQ ID NO: 11 or at least the variable domain thereof or a nucleotide sequence having a sequence identity of at least 80% thereof, and a light chain nucleotide sequence as in SEQ ID NO: 12 or at least the variable domain thereof or a nucleotide sequence having a sequence identity of at least 80% thereof;
c) a heavy chain nucleotide sequence as in SEQ ID NO: 21 or at least the variable domain thereof or a nucleotide sequence having a sequence identity of at least 80% thereof, and a light chain nucleotide sequence as in SEQ ID NO: 22 or at least the variable domain thereof or a nucleotide sequence having a sequence identity of at least 80% thereof;
d) a heavy chain nucleotide sequence as in SEQ ID NO: 31 or at least the variable domain thereof or a nucleotide sequence having a sequence identity of at least 80% thereof, and a light chain amino acid sequence as in SEQ ID NO: 32 or at least the variable domain thereof or a nucleotide sequence having a sequence identity of at least 80% thereof.

Preferred nucleotide sequences of the invention have a sequence identity of at least 85%, more preferably 90%, even more preferably 93%, 94%, 95%, 96%, 97%, 98% or 99%.

The antibody or fragments thereof according to the present invention may be any antibody of natural and/or synthetic origin, an antibody of mammalian origin or a humanized. Preferably, the constant domain, if present, is a human constant domain. The variable domain is preferably a mammalian variable domain, e.g. a humanized or a human variable domain.

Antibodies or fragments thereof according to the invention may be polyclonal or monoclonal antibodies. Monoclonal antibodies are preferred. In particular, the antibodies of the present invention are preferably selected from the group consisting of recombinant antibodies, humanized or fully human antibodies, chimeric antibodies, multispecific antibodies, in particular bispecific antibodies, or fragments thereof. Monoclonal antibodies may be produced by any suitable method such as that of Köhler and Milstein (1975) or by recombinant DNA methods. Monoclonal antibodies may also be isolated from phage antibody libraries using techniques described in Clackson et al. (1991).

Humanized forms of the antibodies may be generated according to the methods known in the art, (Kettleborough C.A. et al., 1991), such as chimerization or CDR grafting. Alternative methods for the production of humanized antibodies are well known in the art and are described in, e.g., EP 0239400 and WO 90/07861. Human antibodies can also be derived by in vitro methods. Suitable examples include but are not limited to phage display, yeast display, and the like.

According the present invention “chimeric antibody” relates to antibodies comprising polypeptides from different species, such as, for example, mouse and human. The production of chimeric antibodies is described, for example, in WO 89/09622.

The term antibody includes “fragments” or “derivatives”, which have at least one antigen binding site of the antibody.

According to a preferred embodiment the antibody or fragment thereof may be a Fab fragment, a Fab′ fragment, a F(ab′)₂ fragment, a Fv fragment, a diabody, a ScFv, a small modular immunopharmaceutical (SMIP), an affibody, an avimer, a nanobody, a domain antibody and/or single chains.

The antibody of the invention may be preferably of the IgG₁, IgG₂, IgG₃, IgG₄, IgM, IgA₁, IgA₂, IgA_{sec}, IgD, and IgE antibody-type. It will be appreciated that antibodies that are generated need not initially possess such an isotype but, rather the antibody as generated can possess any isotype and that the antibody can be isotype-switched.

A further embodiment of the present invention is a vector comprising the nucleic acid coding for the antibody of the invention. Said vector is selected from a phage, a plasmid, a viral or a retroviral vector. Preferably, the vector of the invention is an expression vector wherein the nucleic acid molecule is operatively linked to one or more control sequences allowing the transcription and optionally the expression in prokaryotic and/or eukaryotic host cells.

A further embodiment of the present invention is a host comprising the vector of the invention, selected from a prokaryotic or eukaryotic cell, preferably a mammalian or a human cell, or a non-human transgenic animal.

A further embodiment of the present invention is a method for the preparation of the antibody, or a fragment thereof disclosed above, comprising culturing the host of the invention under conditions that allow synthesis of said antibody and recovering said antibody from said culture.

A further embodiment is an antibody, or a fragment thereof obtained by the method disclosed above.

Preferably, the antibodies of fragments thereof according to the present invention are humanized antibodies.

A further embodiment of the present invention is the use of the aforesaid antibody or a fragment thereof in the treatment of a viral infection caused by a virus classified in the family of Adenoviridae, Anelloviridae, Arenaviridae, Astroviridae, Bornaviridaea, Bunyaviridae, Caliciviridae, Coronaviridae, Filoviridaea, Flaviviridae, Hepadnaviridae, Hepeviridae, Herpesviridae, Orthomyxoviridae, Papillomaviridae, Paramyxoviridaea, Parvoviridae, Picobirnaviridae, Picobirna, Picornaviridae, Pneumoviridae, Polyomaviridae, Poxviridae, Reoviridae, Retroviridae, Rhabdoviridaea, Togaviridae or Deltae.

Preferably said antibody is used in the treatment of a viral infection caused by a virus of the family of Coronaviridae, more preferably the SARS-COV-2 virus or any other virus whose entry in the host cell is related to the presence of IFITM2 or IFITM3 proteins.

A further embodiment of the present invention is a pharmaceutical composition comprising at least one of the aforesaid antibody or a fragment thereof and at least one pharmaceutically acceptable excipient or carrier.

A further embodiment of the present invention is the use of said composition in the treatment of a viral infection caused by a virus classified in the family of Adenoviridae, Anelloviridae, Arenaviridae, Astroviridae, Bornaviridaea, Bunyaviridae, Caliciviridae, Coronaviridae, Filoviridaea, Flaviviridae, Hepadnaviridae, Hepeviridae, Herpesviridae, Orthomyxoviridae, Papillomaviridae, Paramyxoviridaea, Parvoviridae, Picobirnaviridae, Picobirna, Picornaviridae, Pneumoviridae, Polyomaviridae, Poxviridae, Reoviridae, Retroviridae, Rhabdoviridaea, Togaviridae or Deltae.

Preferably, said composition is used in the treatment of a viral infection caused by a virus of the family of Coronaviridae, more preferably the SARS-COV-2 virus or any other virus whose entry in the host cell is related to the presence of IFITM2 or IFITM3 proteins.

The composition of the present invention can be formulated in a form suitable for oral administration or in a form suitable for parenteral or topical administration.

In a preferred embodiment of the present invention, said oral form can be chosen from the following: tablets, capsules, solutions, suspensions, granules or oily capsules.

In a further preferred embodiment of the present invention, said topical form can be chosen from the following: cream, ointment, ointment, solution, suspension, eye drops, pessary, nebulizer solution, spray, powder, or gel.

In a further preferred embodiment of this invention, said parenteral form can be either an aqueous buffer solution or an oily suspension.

Said parenteral administration includes administration by intramuscular, intravenous, intradermal, subcutaneous, intraperitoneal, intranodal, or intrasplenic means.

Preferably the pharmaceutical composition according to the present invention comprises a further active principle, selected from monoclonal antibodies, antiviral drugs as entry blockers, nucleoside/nucleoside analogues and non-nucleoside analogues, IFNs or protease inhibitors.

More preferably said anti-viral drugs are selected from: Amantadine, Rimantadine, Ibalizumab, Enfuvirtide, Vicriviroc, Aciclovir, Valacyclovir, Cidofovir, Foscarnet, Atazanavir, Fosamprenavir, Lopinavir, Darunavir, Nelfinavir, Indinavir, Saquinavir, Ritonavir or Remdesivir.

In a further preferred embodiment the pharmaceutical composition according to the present invention comprises a further active principle, selected from anthelmintic drugs or antimalarial drugs.

Preferably said anthelmintic drugs is Ivermectin.

More preferably said antimalarial drugs is chloroquine or hydroxychloroquine.

### EXAMPLES

### Materials and Methods

Cloning and expression of recombinant IFITM-2 and IFITM-3 NTDs. The NTDs (N-Terminal Domains) of IFITM2 (aa 1-56, SEQ ID N. 40) and IFITM3 (aa 1-57, SEQ ID N. 41) were cloned in pC-AviTag SUMO Vector™ (Lucigen, WI, USA) and expressed in E. coli as fusion protein with biotinylated tag. The expression and production of the proteins were then induced and optimized according to the manufacturer instructions. As expected, the recombinant proteins carried a fused C-terminal biotinylated (Bt) tag. The subsequent cleavage with SUMO Express Protease (Lucigen, #30801-2) allowed to release the Bt-tagged NTDs that were subsequently purified on NTA-Ni resin (Sigma, #P6611) to remove the contaminants (His-SUMO tag and His-tagged protease).

Pierce High-Capacity Endotoxin Removal Spin Column (Pierce, #88274, Waltham, MA, USA) was used to obtain endotoxin-free preparations. Endotoxin concentration was measured by QCL-1000™ Assay (LONZA; #50-647U) following the manufacturer instructions.

### Western Blot Analysis

100 ng of recombinant IFITM2 protein (Proteintech AG #17917) or recombinat IFITM3 protein (Proteintech AG #17863) or IFITM2 NTD peptide or IFITM3 NTD peptide were run on 15% SDS-PAGE gels and electrophoretically transferred to a nitrocellulose membrane.

Nitrocellulose blots were blocked with 10% non-fat dry milk in TBST buffer (20 mM Tris-HCI PH 7.4, 500 mM NaCl, and 0.1% Tween 20) and incubated with primary antibody in TBST containing 5% non-fat dry milk, overnight at 4° C. Immunoreactivity was detected by sequential incubation with HRP-conjugated secondary antibody, (anti-mouse 1:5000, Jackson ImmunoResearch cat. No. 115-035-003; HRP anti-human IgGs 1:20000, Sigma-Aldrich cat. No. A0170,) or HRP-conjugated Avidin (1:1000, eBioscience cat. No. 18-4100-94) and ECL reagents (Pierce cat. No. 32106)

### ELISA Assay for Anti-IFITMs Antibodies

96-well microplates (Thermo Scientific™ MaxiSorp™, cat. no. 442404, Waltham, MA, USA) were coated with 50 μL of solutions containing subsequent peptides or proteins: IFITM2 antigene peptide, IFITM3 antigene peptide, scrambled peptide, recombinant human IFITM1 (Proteintech, Cat. No. Ag2320), recombinant human IFITM2 (Proteintech, Cat. No. Ag17917), recombinant human IFITM3 (Proteintech, Cat. No. Ag17863) (1 μg·mL-1 in PBS1X) and incubated overnight at 4° C. The day after, wells were washed with PBS 1X-0.1% Tween (washing buffer) and the blocking of nonspecific sites was performed for 1 h at room temperature in PBS 1X containing 0.5% fish gelatin (Sigma-Aldrich, Saint Louis, MO, USA). Hence, plates were washed five times with the washing buffer and loaded with different concentrations of anti-IFITMs monoclonal and polyclonal antibodies. Plates were then extensively washed and incubated 30 minutes at room temperature with HRP-conjugated anti-mouse IgGs 1:20000 (Jackson ImmunoResearch, Cambridgeshire, UK, cat. No. 115-035-205) or anti-human IgG 1:20000 (Sigma-Aldrich, cat. No. A0170) or anti-rabbit 1:2000 (Jackson ImmunoResearch, Cambridgeshire, UK Cat. No 111-035-003. Subsequently, TMB solution 1X (eBioscience, San Diego, CA, USA) was added to the wells for the analyte detection. The chromogenic reaction was blocked by acidification with 0.5 m H₂SO₄, and the optical density (O.D.) was measured at 450 nm. Commercially available anti-IFITM2 antibodies tested in ELISA: Abnova, H00010581-M14, Proteintech 66137-1-Ig and Proteintech 12769-1-AP.

### FACS Binding Assay

VERO E6 (ATCC, Cat. No CRL-1586), or Calu-3 (ATCC, Cat. No HTB-55) cells were washed with PBS 1X and treated with Non-Enzymatic Cell Dissociation Solution (ATCC Cat. No. 30-2103™). Cells were harvested by centrifugation and incubated with 80 ul of PBS 1X containing 10% Δ FBS and 0.1% NaN₃ (binding buffer) and 20 μl of FcR Blocking Reagent (Miltenyi Biotec) for 15 min on ice, following manufacturer's instructions (cat. No. 130-059-901). Then, cells (1×10⁶ ml⁻¹ for cell lines) were resuspended in binding buffer and incubated for 15 min on ice. Thereafter, cells were incubated with different concentrations of mAb clone 5D11B9 or mAb clone 3G6D9 or mAb clone 1A12D11 in binding buffer (100 μl) for 30 minutes on ice. Mouse IgG_{2b}-FITC or human IgG₄ were used as negative controls. After incubation, cells were washed three times with PBS 1X containing 2% decomplemented FBS and 0.1% NaN₃ (washing buffer), centrifuged for 10 minutes at 300 g, the bound human monoclonal antibody was revealed by addition 100 μl a 1:50 dilution of PE-conjugated secondary hIgG₄ (SouthernBiotech, Cat. No. 9200-09) and incubated in binding buffer for 30 min on ice. After incubation, cells were washed three times with washing buffer, centrifuged for 10 minutes at 300 g, resuspended in 300 μl of binding buffer and analyzed by flow cytometry.

In binding assays with Biotinylated Recombinant Sars-COV-2 Spike protein (R&D Systems, Cat. No BT10549) on Vero E6 cell surface, the cells were incubated with different concentrations of protein (10, 5, 2.5 and 1.25 μg ml⁻¹) in 100 μl binding buffer, 1 hour at 37° C. After incubation, cells were washed three times with washing buffer, centrifuged for 10 minutes at 300 g and revealed by addition 100 μl a dilution 1:200 of PE-Streptavidin (SouthernBiotech cat. No. 7100-09M) in binding buffer for 30 min on ice. After incubation, cells were washed three times with washing buffer, centrifuged for 10 minutes at 300 g, resuspended in 300 μl of binding buffer and analyzed by flow cytometry.

### Neutralization Assay by FACS

Biotinylated Recombinant Sars-COV-2 Spike protein (10 ug ml⁻¹) was pre-incubated 30 minutes at room temperature with patient samples of human serum containing SARS-COV-2 neutralizing antibodies (1:30) in binding buffer (50 μl). The mix was added to 50 μl of cells suspension (1×10⁵ cells) and, after 1 hour of incubation at 37° C., cells were washed three times with washing buffer and centrifuged for 10 minutes at 300 g. The bound Biotinylated Recombinant Sars-COV-2 Spike protein was revealed by addition 100 μl of PE-Streptavidin (1:200) and incubated in binding buffer for 30 min on ice. After incubation, cells were washed three times with washing buffer, centrifuged for 10 minutes at 300 g, resuspended in 300 μl of binding buffer and analyzed by flow cytometry.

Vero E6 cells were pre-incubated with mAb clone 5D11B9 or mAb clone 3G6D9 and mAb clone 1A12D11) (30 ug ml⁻¹) in binding buffer (50 μl) 30 minutes at on ice. Then, the Biotinylated Recombinant Sars-Cov-2 Spike protein (10 ug ml⁻¹) was added in binding buffer (50 μl) and incubated 1 hour at 37° C. After washing three times with washing buffer, was added 100 μl of PE-Streptavidin (1:200) in binding buffer and incubated for 30 min on ice. After incubation, cells were washed with washing buffer, centrifuged for 10 minutes at 300 g, resuspended in 300 μl of binding buffer and analyzed by flow cytometry.

### Peptide Competition Assay by FACS

mAb clone 5D11B9 or mAb clone 3G6D9 and mAb clone 1A12D11) (20 ug ml⁻¹), were pre-incubated 30 minutes at room temperature with the IFITM2 antigene peptide or IFITM3 antigene peptide at different concentrations (1X, 10X and 20X) in binding buffer (50 μl). The mix was added to 50 μl of cells suspension (1×10⁵ cells) and, after 30 min of incubation on ice, cells were washed with washing buffer and centrifuged for 10 minutes at 300 g. The bound human monoclonal antibody was revealed by addition a PE-conjugated secondary hIgG₄ (1:50) and incubated in binding buffer for 30 min on ice. After incubation, cells were washed with washing buffer, centrifuged for 10 minutes at 300 g, resuspended in 300 μl of binding buffer and analyzed by flow cytometry.

### Plaque Assay

Vero E6 cells were plated in 12 multiwells (2×105 cells/well). After 24 hours, cells at 80% confluence were treated with each Ab and simultaneously infected with the SARS-COV-2 (at a viral titer of 5×10³ PFU/mL) or other types of viruses, with DNA genome i.e. HSV-1 (Herpes Simplex Virus-1 and HSV-2 (Herpers Simplex Virus-2). After the adsorption time of 2h, time necessary for the virus to take root and enter the host cells, plates were washed with 1X PBS, and a mixture of 3% carboxymethylcellulose/10% FBS 1:3 culture medium was administered. After 48 h, the cytopathic effect is first observed under the microscope and then plates are stained with Crystal Violet 0.5%/Formaldehyde 4%. Plaques are counted under the microscope and viral inhibition is calculated against the untreated virus control according to the formula:

100−[(treated well plaques/plaques in infected control)×100].

The experimental conditions were different only for HCoV-OC43 (virus with RNA genome-human betacoronavirus). In detail, 2×10⁴ Vero cells/well are seeded in 96-well plates and incubated for 24 hours. Cell monolayers are then infected with 100 μl at a viral titer of 5×102 PFU/ml. At the same time, 100 μl of medium, without or with serial dilutions of each Ab, were added. After 3 days of incubation at 37° C., cell viability is determined by the MTT assay and cythopathic effect (CPE) was analyzed. Commercially available anti-IFITM2 antibodies tested in the plaque assay were: Abnova, H00010581-M14, Proteintech 66137-1-Ig and LS-C322156.

### RT-PCR

Total RNA was isolated using TRIzol reagent and quantified by measuring the absorbance at 260/280 nm (NanoDrop 2000, Thermo Fisher Scientific, Waltham, MA, USA). Approximately 1 μg of total RNA was reverse-transcribed to cDNA by 5X All-In-One RT MasterMix (Applied Biological Materials, Richmond, Canada). Quantitative polymerase chain reaction was run in triplicate using a CFX Thermal Cycler (Bio-Rad, Hercules, CA, USA). 2 μl of cDNA were amplified in 20 μl reactions using BrightGreen 2X qPCR MasterMix-No Dye (Applied Biological Materials, Richmond, Canada) and 0.1 μM of primer. Relative target Ct (the threshold cycle) values of the spike protein(S) was normalized to Glyceraldehyde 3-phosphate dehydrogenase (GAPDH), as housekeeping gene. The mRNA levels of cells treated with antibodies, or with IFITM2-specific siRNAs, were expressed using the 2-^{ΔΔCt} method. Primers used for qPCR:

| GENE | FORWARD PRIMER | REVERSE PRIMER |
|---|---|---|
| Spike | AGG TTG ATC ACA | GCT GAC TGA GGG |
| | GGC AGA CT | AAG GAC |
| | (SEQ ID NO: 42) | (SEQ ID NO: 43) |
| GAPDH | CCT TTC ATT GAG | CGT ACA TGG GAG |
| | CTC CAT | CGT C |
| | (SEQ ID NO: 44) | (SEQ ID NO: 45) |
| Human | ACG GAA CTA CTG | CAC TTC CTG CTC |
| IFITM2 | GGG AAA GG | CTC CTT GA |
| | (SEQ ID NO: 46) | (SEQ ID NO: 47) |
| Human | TGA CCC AGA TCA | AGG GCA TAC CCC |
| Beta | TGT TTG AGA | TCG TAG AT |
| Actin | (SEQ ID NO: 48) | (SEQ ID NO: 49) |

Cycling program used for qPCR:

| Step | Temperature | Duration | | Cycle(s) |
|---|---|---|---|---|
| Enzyme activation | 95° C. | 10 | mins | 1 |
| Denaturation | 95° C. | 15 | secs | |
| Annealing/Extension | 60° C. | 60 | secs | 40 |

| | |
|---|---|
| Melting curve | Refer to specific guidelines for instrument used |

### Recombinant Antibodies Production

Sequences coding for the antibodies were cloned in Evi-5 expression vector (Evitria AG, Switzerland) and expressed in CHO-K1 cells.

For antibody chimerization, the murine constant regions were replaced with the human constant regions.

### siRNA Transfection

Twenty-four hours after seeding into 12-well cell culture plates (2×10⁵ cells/ml cells for each well) or 6-well cell culture plates (4×10⁵ cells/ml cells for each well) in complete medium, Calu-3 cells were transfected with 25 nM of non-targeting siRNA or IFITM2 specific siRNA (IFITM2 N-Terminal siRNA: 5′-AACCUUCUCUCCUGUCAACAG-3′ (SEQ ID NO: 50), 3′-UUGGAAGAGAGGACAGUUGUC-5′ (SEQ ID NO: 51); IFITM2 C-Terminal siRNA: 5′-UUGGUCGUCCAGGCCCAGCGU-3′ (SEQ ID NO: 52), 3′-AACCAGCAGGUCCGGGUCGCA-5′ (SEQ ID NO: 53),) using TransiT X2 Dynamic Delivery System (Mirus Bio LLC, Cat. No. MIR 6005) according to the manufacturer's instructions. Twenty-four hours after transfection, cells were harvested for RNA extraction and qRT-PCR using specific primer for human IFITM2.

Forty-eight hours after transfection cells were collected and stained with monoclonal antibody 5D11B9 FITC-conjugated for binding on Calu-3 cell surface by flow cytometry.

### ACE-2 Enzimatic Activity Assay

The catalytic activities of endogenous ACE2 was detected by ACE2 Activity Assay Kit Fluorometric (Abcam, Cat. No ab273297).

Vero E6 and Calu-3 cells were seeded in a 96-well plate at 1×10⁵ cells/well and cultured overnight. The cells were then incubated with 30 μg ml⁻¹ monoclonal antibody clone 5D11B9, 10 μg ml⁻¹ polyclonal Goat antibody ACE2 (R&D Systems, Minneapolis, Cat. No AF933) and isotype (mouse IgG2b and Normal Goat IgG) for 1and 24 hours at 37° C. Cells were then washed with 1X PBS before ACE2 Activity Assay in according to the manufacturer's instructions. Cells were incubated for 30 min at 37° C. and then transferred to black 96-well plate for fluorescence reading (320/420 nm).

### Confocal Microscopy

Calu-3 cells cultured under standard growth conditions were plated on glass coverslips at 70% of confluence and were allowed to grow for 24 h at 37° C. in 5% CO2. Cells were then incubated 2 hours at 4° C. after that, recombinant Spike protein (ProSci 97-092) was added to the culture media at the concentration of 5micrograms/ml together with the 5D11B9 mAb (15 micrograms/ml) or with an unrelated murine IgG2b (15 micrograms/ml). After one additional hour at 4° C. cells were incubated at 37° C. and harvested after 5 and 30 minutes. At the end of treatments, cells were washed once with PBS 1X, fixed with 3.7% formaldehyde in PBS, for 20 minutes, at room temperature (RT), washed in 0.1M glycine and then permeabilized with 0,005% saponin in PBS 1X/BSA 3% for 30 minutes at RT. Then, in order to detect the spike recombinant fused protein, cells were incubated with an anti-mouse antibody (southern Biotech 1031-32) 1:200 for 1 hour at RT. Coverslips were mounted with Prolong Gold Antifade Reagent as well as DAPI (diamidino-2-phenylindole) (Thermo Fisher Scientific, Inc., MA, USA) to visualize nuclei. Slides were then coverslipped using an aqueous mounting medium and analyzed using a confocal laser scanning microscope (Leica SP5, Leica Microsystems, Wetzlar, Germany). Images were acquired in sequential scan mode by using the same acquisitions parameters (laser intensities, gain photomultipliers, pinhole aperture, x40 objective) when comparing experimental and control material. For figures preparation, brightness and contrast of images were adjusted by taking care to leave a light cellular fluorescence background, for visual appreciation of the lowest fluorescence intensity features and to help comparison among the different experimental groups.

### Example 1

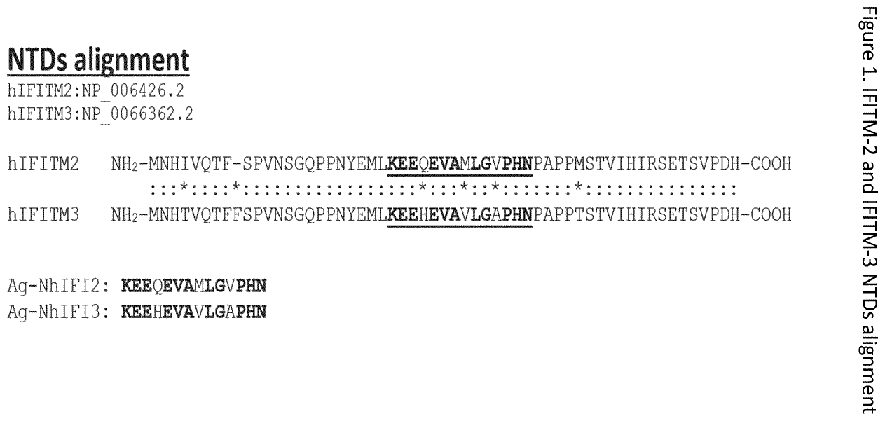

We analyzed the N-Terminal (NTD) sequences of IFITM2 and IFITM3 and selected short sequence stretches encompassing the highest number of amino acidic substitutions between the two sequences to synthesize oligopeptides for mice immunization. Sequence alignment and antigen sequences (SEQ ID N. 38 and SEQ ID N. 39) used to immunize the mice are reported in FIG. 1

### Example 2

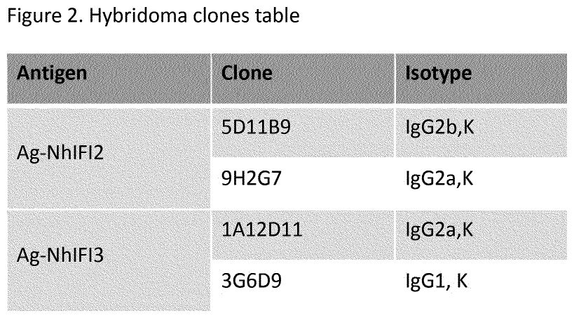

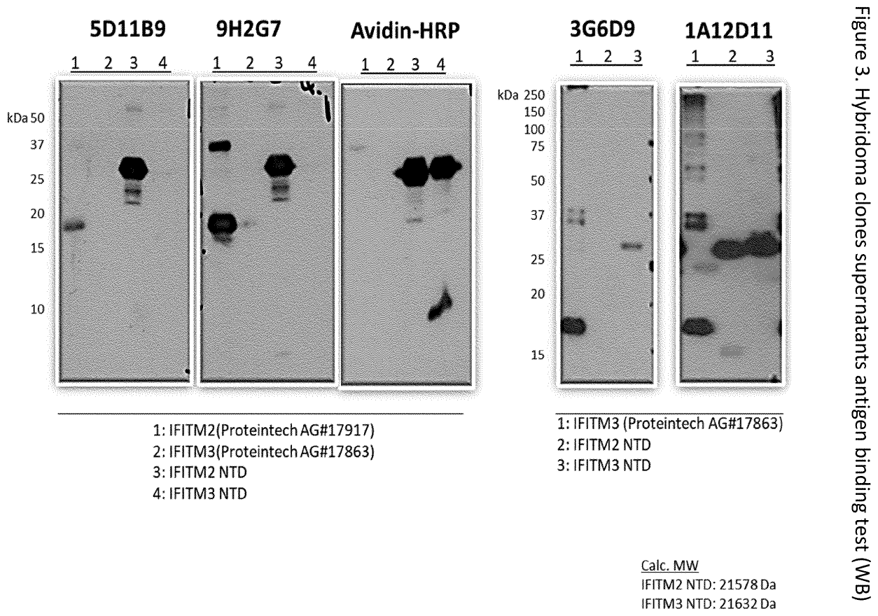

Hybridoma clones and murine IgG isotypes are described in FIG. 2 and obtained as described by Kohler at al., 1975.

### Example 3

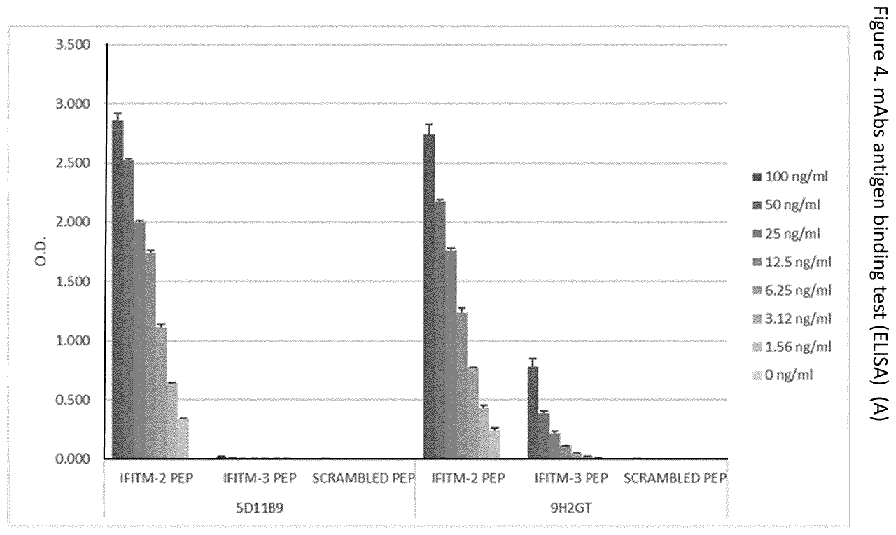

The IFITM2 and IFITM3 NTD recombinant proteins were used in the preliminary characterizations by Western blot analyses of hybridoma clones' supernatants identified and are represented in FIG. 3. Clones 5D11B9 and 9H2G7 showed selectivity towards IFITM-2 protein, clone 3GD9 showed selectivity towards IFITM-3 protein, while clone 1A12D11 reacted with both IFITM2 and IFTM3 proteins.

### Example 4

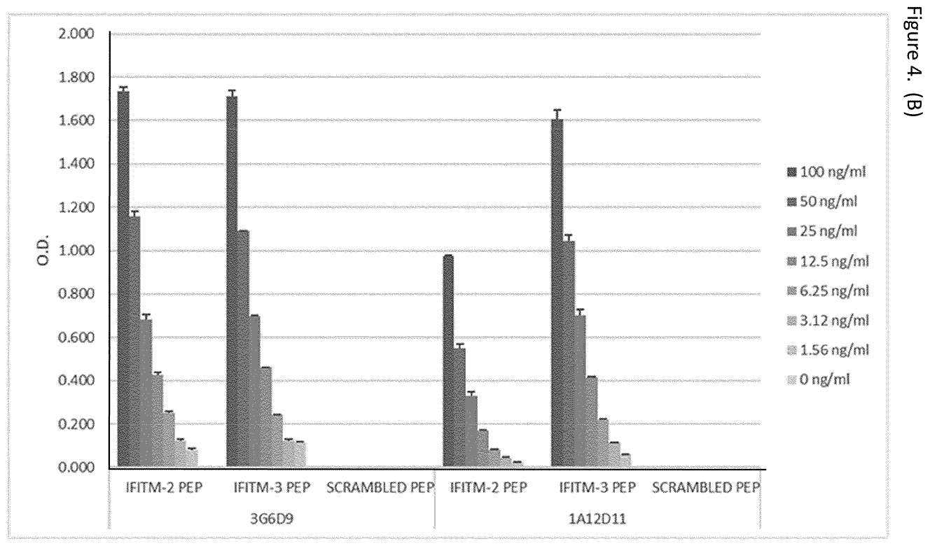

The recombinant proteins described in Example 3 were also used in ELISA assays on purified and/or recombinant antibody clones and are represented in FIG. 4. mAb clone 5D11B9 showed selectivity towards IFITM2 protein, while mAb clones 9H2G7, 3G6D9 and 1A12D11 reacted with both IFITM2 and IFITM3 proteins. Results are expressed in O.D. means.

### Example 5

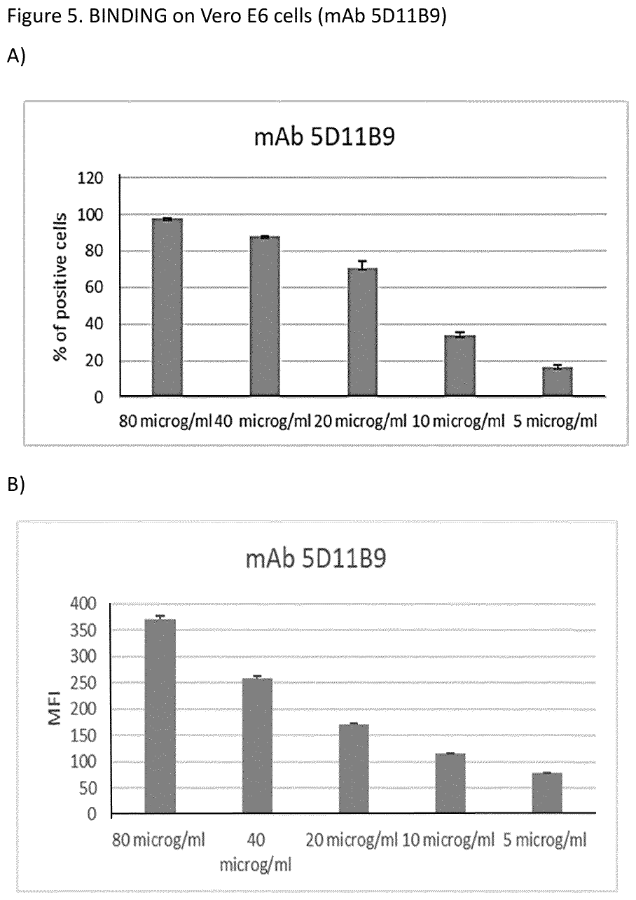

The monoclonal antibody 5D11B9 was FITC-conjugated and tested for binding to Vero E6 cell surface by flow cytometry. In FIG. 5 are showed the results of mAb titration from 80 micrograms to 5 micrograms per ml as A) percentage of positive cells and B) Mean Fluorescent Intensity (mean±standard deviation).

### Example 6

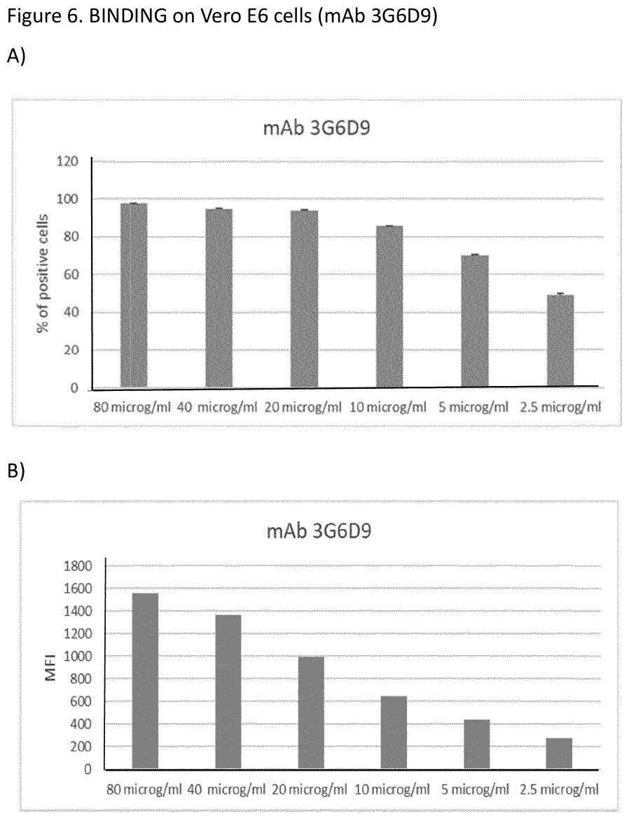

The monoclonal antibody 3G6D9 was sequenced by standard techniques and produced as recombinant antibody in CHO cells as a human IgG₄ chimera. The purified antibody was tested in flow cytometry for binding to the Vero E6 cell surface using a PE-conjugated anti-human IgG4 antibody as a secondary revealing antibody. In FIG. 6 are shown the results of mAb titration from 80 micrograms to 2.5 micrograms per ml as A) percentage of positive cells and B) Mean Fluorescent Intensity (mean±standard deviation).

### Example 7

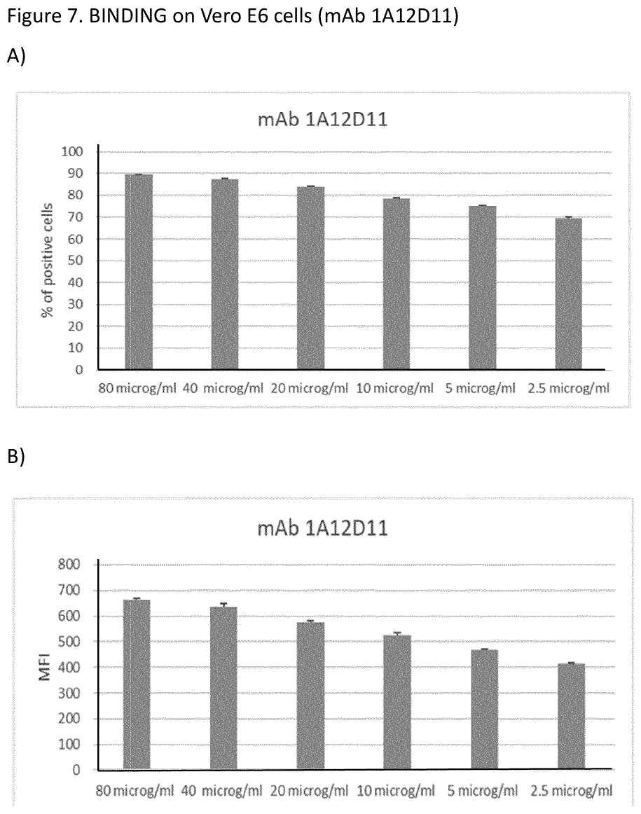

The monoclonal antibody 1A12D11 was sequenced by standard techniques and produced as recombinant antibody in CHO cells as a human IgG₄ chimera. The purified antibody was tested in flow cytometry for binding to the Vero E6 cell surface using a PE-conjugated anti-human IgG4 antibody as a secondary revealing antibody. In FIG. 7 are showed results of mAb titration from 80 micrograms to 2.5 micrograms per ml as A) percentage of positive cells and B) Mean Fluorescent Intensity (mean±standard deviation).

### Example 8

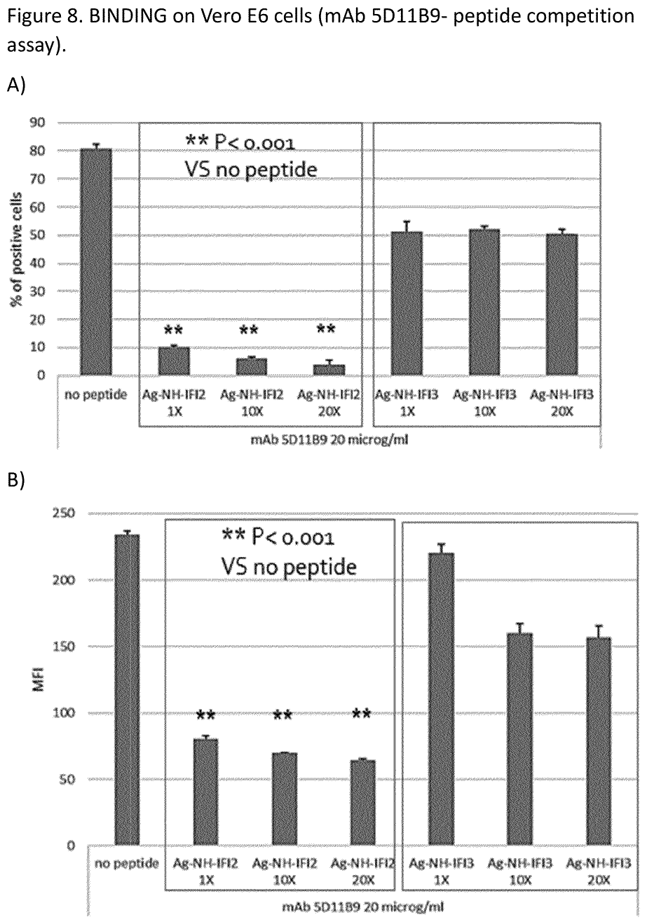

The monoclonal antibody 5D11B9 was tested in flow cytometry for binding to the Vero E6 cell surface in the presence of competing peptides (IFITM-2 and IFITM-3 antigens). In FIG. 8 are showed the results of the peptide competition assay. The figure shows that IFITM2 but not IFITM3 antigens are able to inhibit mAb binding on Vero E6 cell surface. Results are represented as A) percentage of positive cells and B) Mean Fluorescent Intensity (mean±standard deviation). A two-tailed t-test was performed between indicated groups.

### Example 9

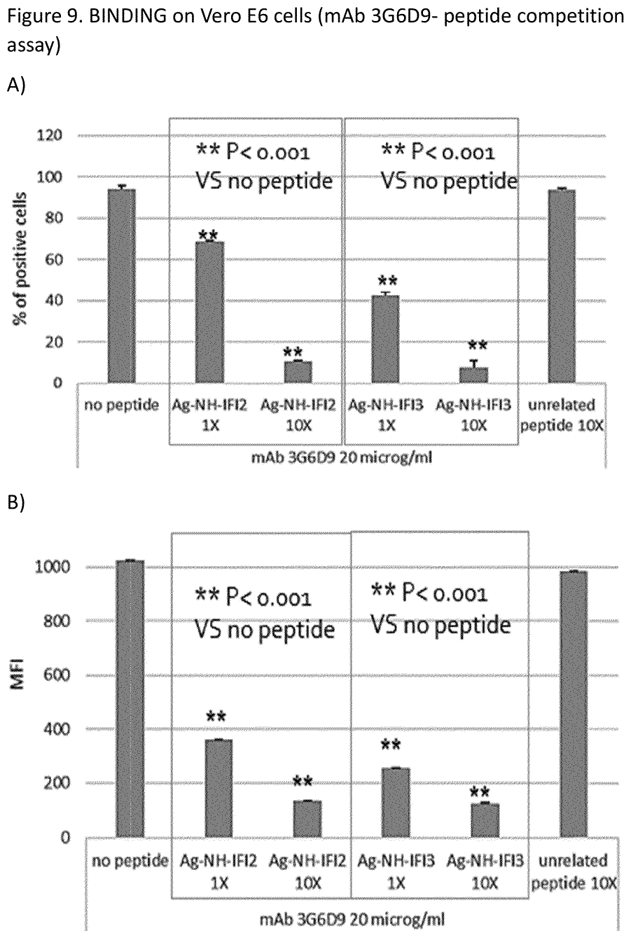

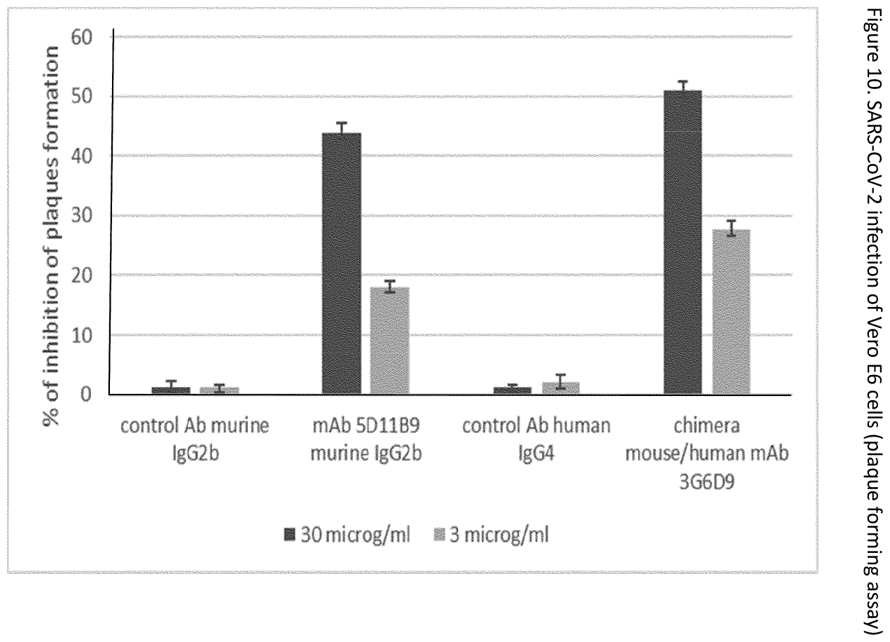

The monoclonal antibody 3G6D9 was tested in flow cytometry for binding to the Vero E6 cell surface in the presence of competing peptides (IFITM2 and IFITM3 antigens). In FIG. 9 are shown the results of peptide competition assay where both IFITM2 and IFITM3 antigens are able to inhibit mAb binding on Vero E6 cell surface. Results are represented as A) percentage of positive cells and B) Mean Fluorescent Intensity (mean±standard deviation). A two-tailed t-test was performed between indicated groups.

### Example 10

The monoclonal antibodies 3G6D9 (murine/human chimera IgG₄) and 5D11B9 (murine IgG_{2b}) were tested for their ability to inhibit SARS-COV-2 infection in Vero E6 cells.

In FIG. 10 the results are shown as % of inhibition of plaque formation formation (mean±standard deviation) The co-administration of monoclonal antibody 5D11B9 and 3G6D9 with genuine SARS-COV-2 inhibited by more than 40% the plaques' number, while an unrelated murine IgG_{2b} or unrelated human IgG₄ did not show any effect.

### Example 11

Vero cells were plated into 12-well cell culture plates (2×105 cells/ml cells for each well) in culture medium. The next day, the monolayer was pre-treated with monoclonal antibody 3G6D9, monoclonal antibody 5D11B9 or unrelated human IgG₄ (30 μg/ml) at 37° C. for 2 hours, then Vero cells were infected with SARS-COV-2 (VR PV10734) at multiplicity of infection of 0.5. After 6 hours post-infection, cells were washed with Phosphate Buffered Saline (PBS) 1X and overlaid with carboxymethylcellulose 0.5% mixed to Dulbecco's Modified Eagle's Medium (DMEM). After 48 hours, total RNA was obtained and used for spike gene detection in cultures.

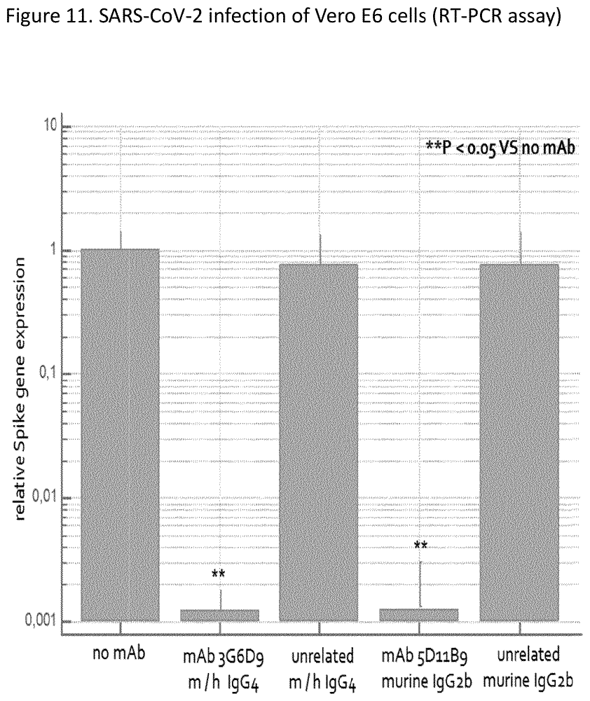

In FIG. 11 the results are shown as relative spike gene expression. Vero cells pre-treatment with mAb 3G6D9 or mAb 5D11B9 (30 μg/ml) abated spike gene expression while an unrelated human IgG₄ did not show any effect.

The results thus demonstrate that both mAb 3G6D9 and mAb 5D11B9 are able to inhibit the SARS-Cov2 infection.

### Example 12

IFITM2 antigen peptide, IFITM3 antigen peptide, scrambled peptide, recombinant human IFITM1, recombinant human IFITM2, recombinant human IFITM3 were used in ELISA test. 5D11B9 mAb binding activity was compared to commercially available anti-IFITM2 antibodies Abnova H00010581-M14, Proteintech 66137-1-Ig and Proteintech 12769-1-AP. mAb clone 5D11B9 (A) showed selectivity towards IFITM-2 peptide and human recombinant protein IFITM2; Abnova H00010581-M14 (B), Proteintech 66137-1-Ig (C) showed mixed selectivity toward IFITM2 and IFITM3 proteins; Proteintech 12769-1-AP (D) showed aspecific binding with all IFITMs human recombinant protein tested. Results were expressed in O.D. mean.

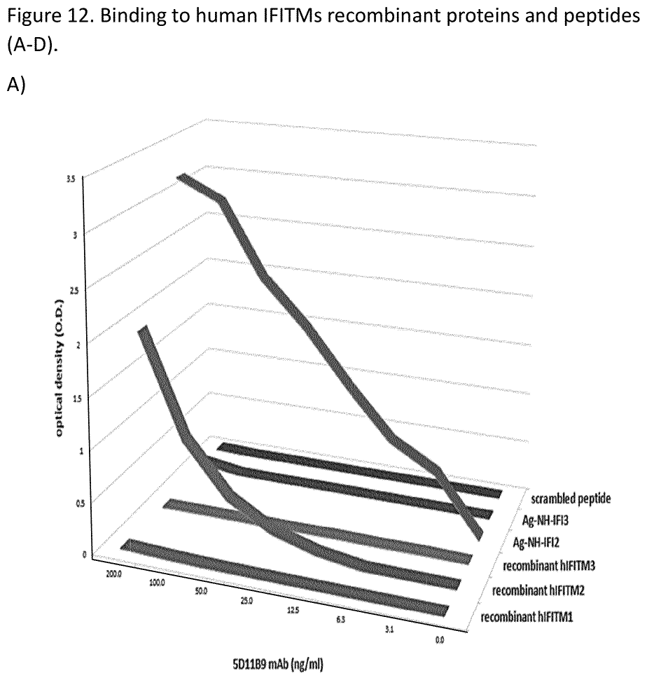

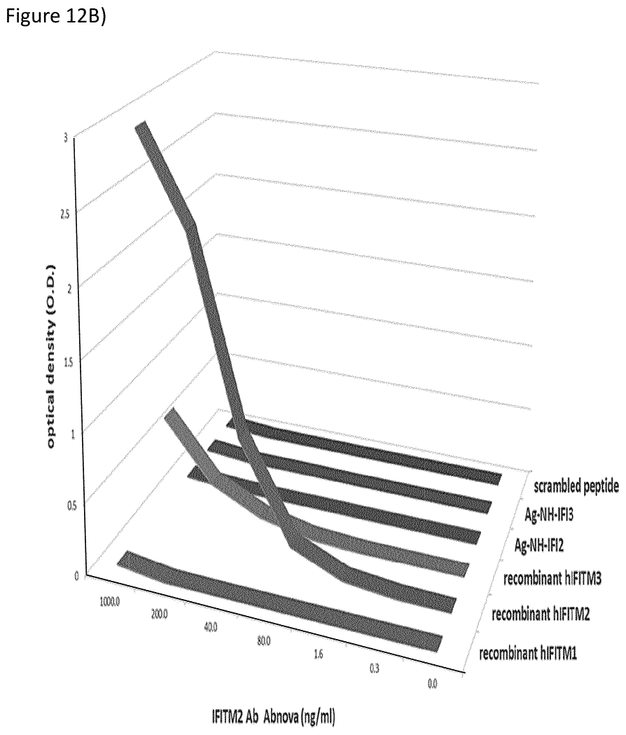

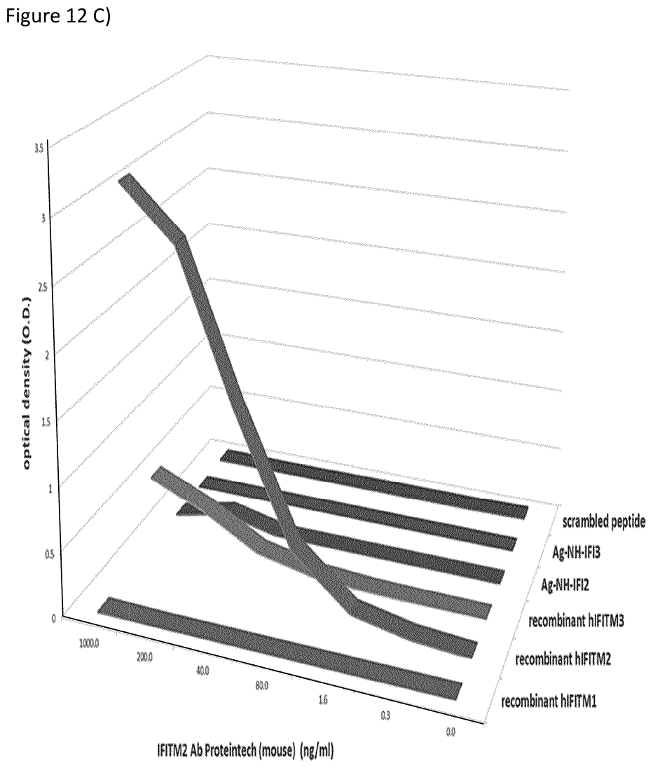

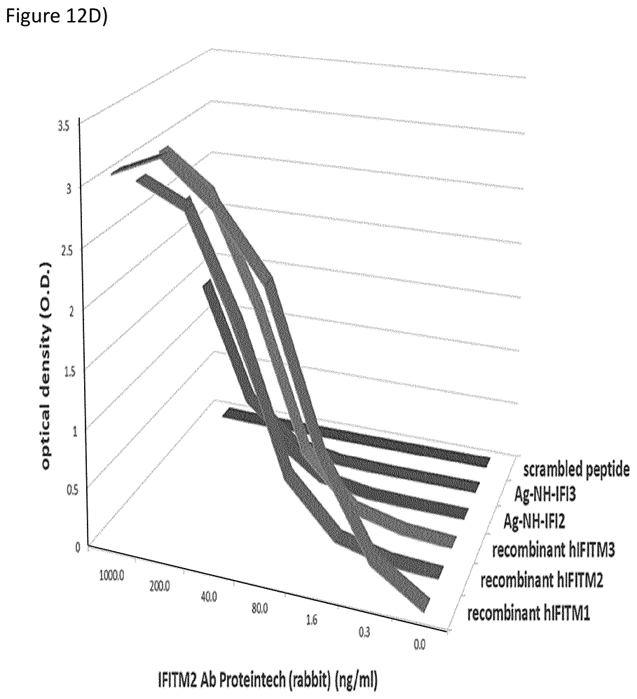

The obtained results (see FIG. 12A-D) clearly demonstrate that the monoclonal antibody 5D11B9 has an higher affinity towards the IFITM-2 peptide and that said antibody is more specific than the commercial antibodies tested.

### Example 13

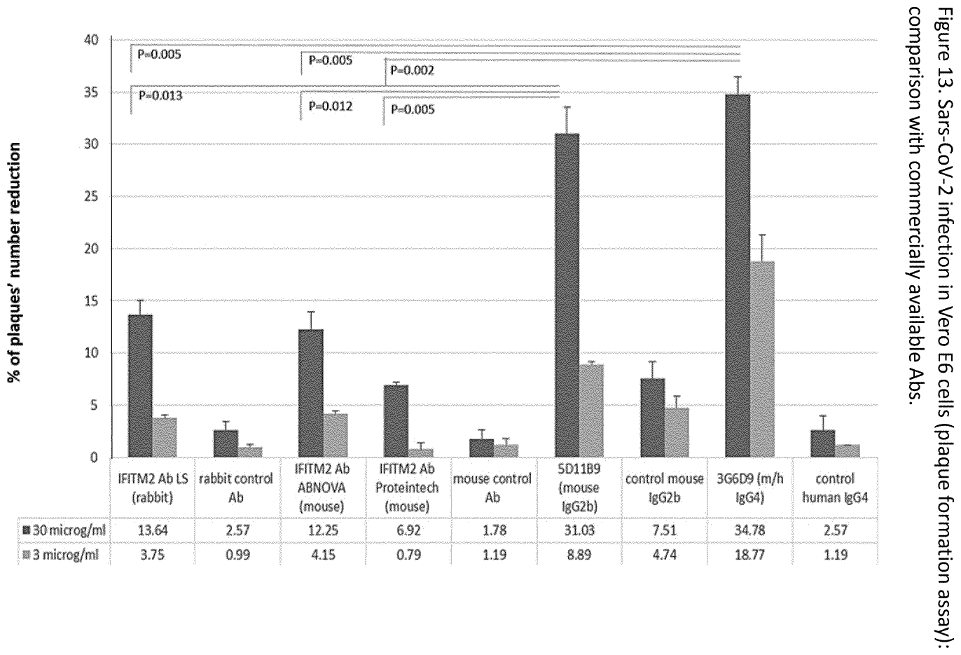

The monoclonal antibodies 3G6D9 (murine/human chimera IgG4) and 5D11B9 (murine IgG2b) were tested for their inhibitory effects of SARS-COV-2 infection in Vero E6 cells, and compared with the efficacy of commercially available anti-IFITM2 antibodies Abnova, H00010581-M14, Proteintech 66137-1-Ig and LS-C322156. In FIG. 13 the results are shown as percent (%) of inhibition of plaque formation (mean±standard deviation). A two-tailed t-test was performed between indicated groups. The co-administration of monoclonal antibody 5D11B9 or 3G6D9 with genuine SARS-COV-2 inhibited by more than 30% the number of formed plaques, while an unrelated murine IgG2b or unrelated human IgG4 were unable to inhibit the viral infection. A compared assay with commercially available anti-IFITM2 antibodies showed that both polyclonal and monoclonal antibodies barely resulted in inhibiting plaques' number by less than 15%. This demonstrated the higher efficacy of the antibodies of the present invention.

### Example 14

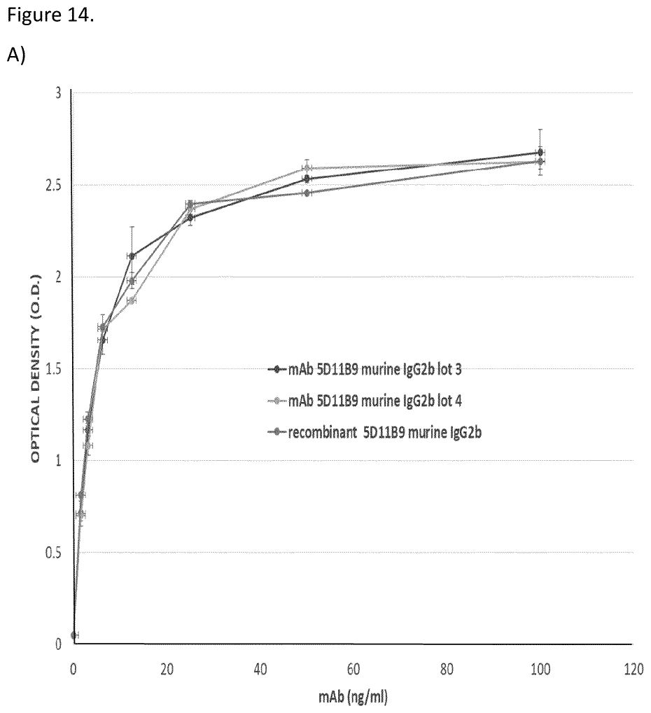

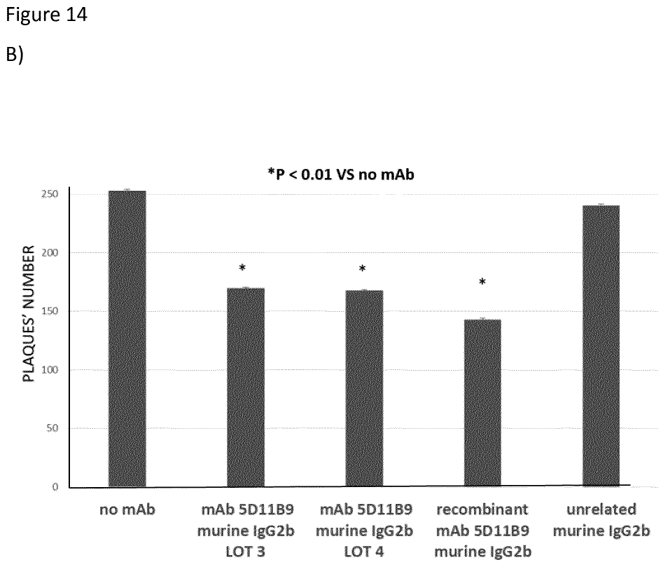

The monoclonal antibody 5D11B9 was sequenced by standard techniques and produced as recombinant antibody in CHO cells as a murine IgG_{2b}. The resulting antibody was tested in comparison to two different lots (lot 3 and lot 4) of the antibody obtained from the hybridoma clone in an antigen-directed ELISA test (FIG. 14A) and in a plaque formation assay in Vero E6 cells (FIG. 14B). In FIG. 14A the results are shown O.D. means (±standard deviation), in FIG. 14B results are shown as plaques' number means (±standard deviation). A two-tailed t-test was performed between indicated groups. The recombinant 5D11B9 showed similar efficiency with respect to the hybridoma monoclonal antibody, demonstrating that variable antibody's sequence described in the present invention and produced as a recombinant protein conserved similar characteristics in respect to monoclonal antibodies naturally produced by the 5D11B9 clone.

### Example 15

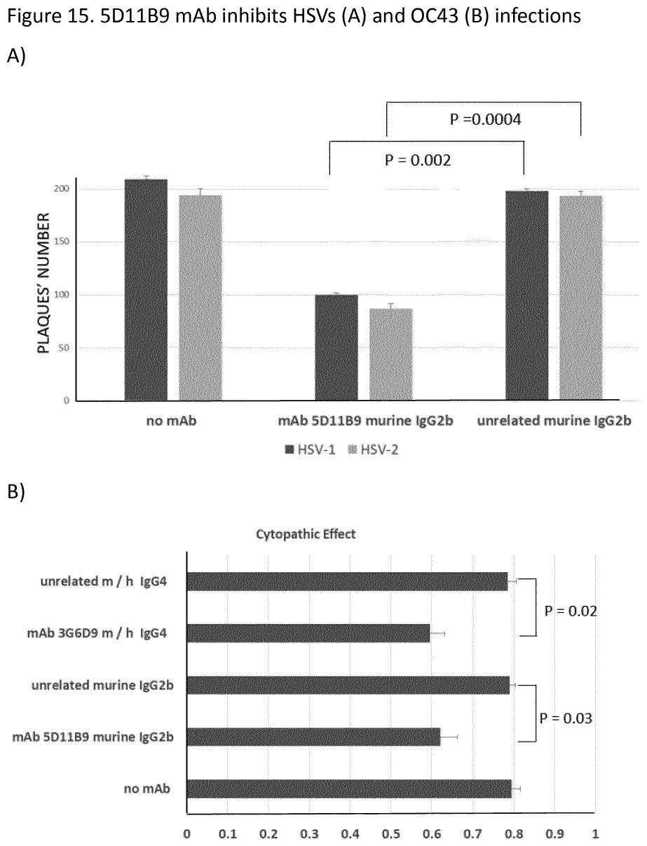

The monoclonal antibody 5D11B9 (murine IgG2b) was tested for its ability to inhibit HSV-1, HSV-2 (Herpes Simplex Virus 1 and 2), In FIG. 15A the results are shown as plaques' number means (±standard deviation). A two-tailed t-test was performed between indicated groups. The co-administration of monoclonal antibody 5D11B9 with genuine HSV-1 or HSV-2 inhibited by more than 50% the plaques' number formation, while an unrelated murine IgG2b did not show any effect. B) The monoclonal antibodies 3G6D9 (murine/human chimera IgG₄) and 5D11B9 (murine IgG_{2b}) were also tested for their ability to inhibit OC43 (beta-coronavirus) infection. showed a 20% inhibition of OC43-induced plaque formation. In FIG. 15B the results are shown as percentage of cytopathic effect (±standard deviation). A two-tailed t-test was performed between indicated groups. The co-administration of monoclonal antibodies 5D11B9 and 3G6D9 with genuine OC-43 virus inhibited its cytopathic effect from 80 to about 60%, while unrelated murine IgGs did not show any effect.

### Example 16

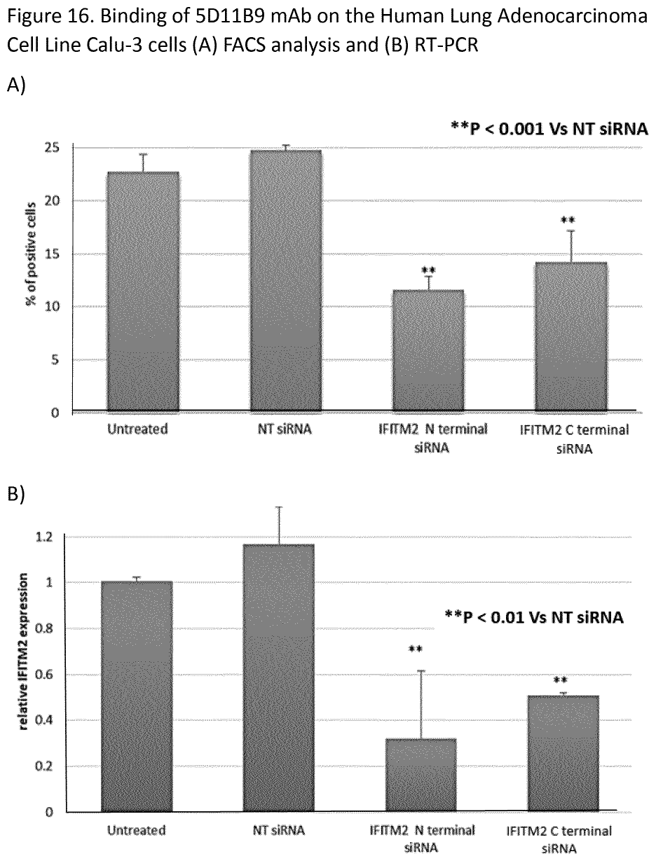

Calu-3 were plated into 6-well or 12 well cell culture plates (4×10⁵ cells/ml or 2×10⁵ cells/ml cells for each well respectively) in complete medium. After 24 hours, cells were transfected with 25 nM of non-targeting siRNA or IFITM2 specific siRNA (IFITM2 N-Terminal and IFITM2 C-Terminal siRNA). After 48 hours, cells were harvested and stained with FITC-conjugated monoclonal antibody 5D11B9, then analyzed by flow cytometry for binding to Calu-3 cells surface. In FIG. 16A the results are shown as percent (%) of positive cells means (±standard deviation). After 24 hours, total RNA was extracted and the expression of IFITM2 analyzed by qRT-PCR. The results of the IFITM2 relative expression are shown in FIG. 16 B. A two-tailed t-test was performed between indicated groups. The experiment showed that the 5D11B9 mAb binding on human Calu-3 cell surface is reduced by siRNAs mediated silencing (FIG. 16A). IFITM2 specific silencing was also demonstrated by a reduced IFITM2 RNA production (FIG. 16B).

### Example 17

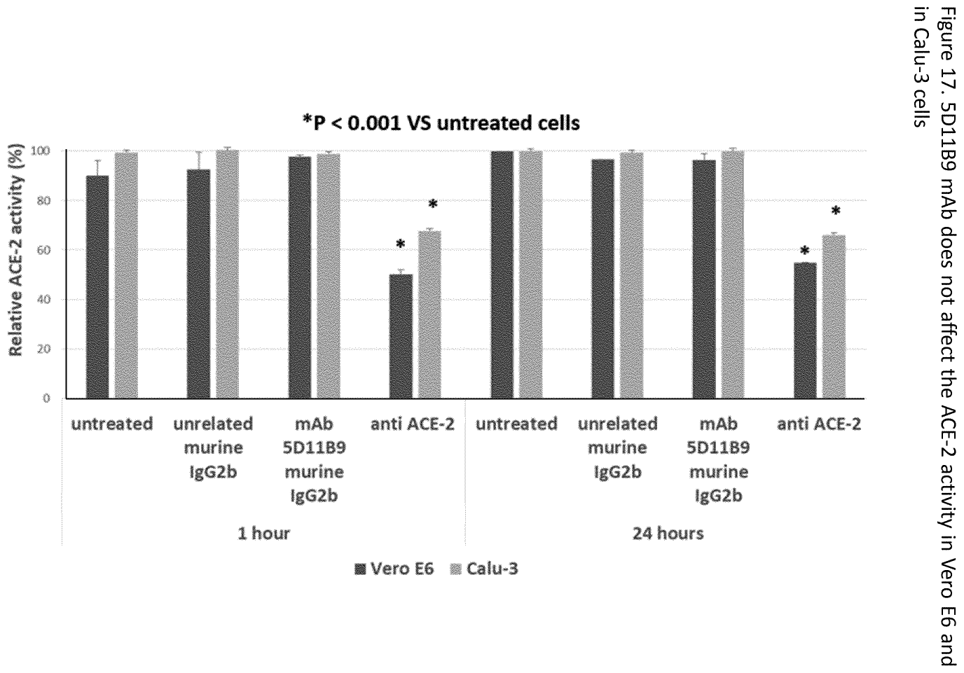

Vero E6 and Calu3 cells were seeded in a 96-well plate at 1×10⁵ cells/well and after an overnight incubation at 37° C. cells were treated with mAb 5D11B9 murine IgG_{2b} and an unrelated murine IgG_{2b} (30 μg ml⁻¹) for 1 and 24 hours at 37° C. ACE-2 polyclonal Goat antibody (10 μg ml⁻¹) was used as positive control. Cells were then washed with 1X PBS before ACE-2 Activity Assay in according to the manufacturer's instructions. Cells were incubated for 30 min at 37° C. and transferred to black 96-well plate for fluorescence reading (320/420 nm). In FIG. 17 (VERO E6 and Calu-3 cells) the results are shown as Relative ACE2 activity (%) means (±standard deviation). A two-tailed t-test was performed between indicated groups.

The treatment with monoclonal antibody clone 5D11B9 did not affect the enzymatic activity of the endogenous ACE-2 on the surface of cells Vero E6 and Calu-3. Said results therefore demonstrate that, while the anti-ACE-2 antibody, directed against the human receptor for Sars COV-2 Spike protein, inhibit the physiological activity of a central human body homeostatic enzyme, the anti-IFITM2 mAb 5D11B9, do not perturb ACE-2 enzymatic activity on both tested cell lines.

### Example 18

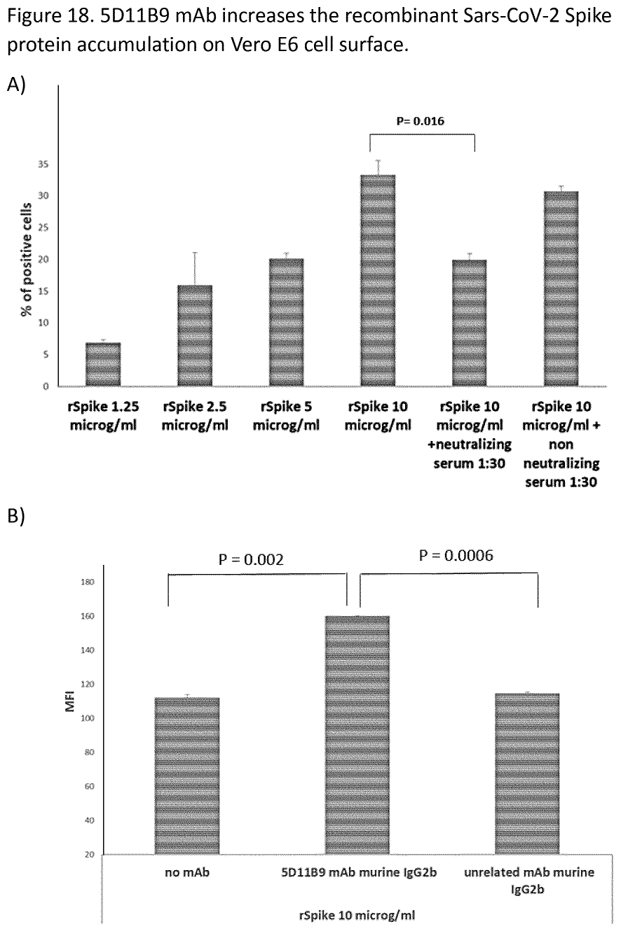

Flow cytometry assays were performed to test the binding of the recombinant biotinylated Spike protein to Vero E6 cell surface using a PE-conjugated streptavidin. FIG. 18A shows the results, as percentage of positive cells, the spike titration (concentrations:1.25 micrograms to 10 micrograms per ml) in cell binding assay. Spike binding to cell surface was efficiently inhibited by a neutralizing serum containing anti-spike antibodies. In panel B, Spike binding to cell surface was measured in the presence of monoclonal antibody 5D11B9. The presence of 5D11B9 significantly increased the signal of the spike binding on Vero E6 cells, thus suggesting its accumulation on the external side of the cell surface.

### Example 19

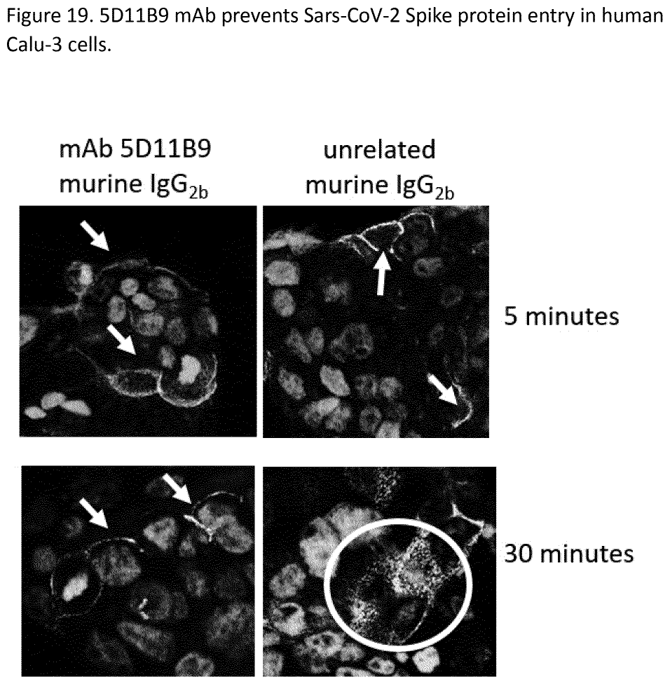

Calu-3 cells were treated with a Sars-COV-2 spike recombinant protein in the presence of the monoclonal antibody 5D11B9 or an unrelated murine IgG_{2b} at 1 hour at 4° C. Cells were then incubated at 37° C. and harvested after 5 and 30 minutes and the signal from the spike protein was detected by immunofluorescence and confocal microscopy. The images displayed in the FIG. 19 showed that after 5 minutes at 37° C. the spike protein accumulate on the cells plasmamembrane (indicated with arrows) in both the two treatment group. After 30 minutes spike protein signal is diffuse in the cytoplasm (indicated with a circle) in cells treated with an unrelated IgG. In the same time point, cells treated with the 5D11B9 mAb, did not show a cytoplasmic spike signal, instead it appears to be confined in the plasmamembrane. The experiment showed that the treatment with the 5D11B9 mAb inhibits Sars-COV-2 spike entry in the target host cells.

### REFERENCES

Bailey C C, Zhong G, Huang I-C, Farzan M. IFITM-Family Proteins: The Cell's First Line of Antiviral Defense. Annu. Rev. Virol. 2014;1(1):261-283.
Brass AL, Huang I, Benita Y, et al. IFITM Proteins Mediate the Innate Immune Response to Influenza A H1N1 Virus, West Nile Virus and Dengue Virus. Genome. 2009;139(7):1243-1254.
Cimolai N. Complicating Infections Associated with Common Endemic Human Respiratory Coronaviruses. Health Secur. 2020 Nov. 11.
Clackson T, Hoogenboom H R, Griffiths A D, Winter G. Making antibody fragments using phage display libraries. Nature 1991; 15;352(6336):624-8.
Friedman R L, Manly S P, McMahon M, Kerr I M, Stark G R. Transcriptional and posttranscriptional regulation of interferon-induced gene expression in human cells. Cell. 1984;38(3):745-755.
Hu, B., Guo, H., Zhou, P. et al. Characteristics of SARS-COV-2 and COVID-19. Nat Rev Microbiol (2020).
Huang, I. C. et al. Distinct patterns of IFITM-mediated restriction of filoviruses, 600 SARS coronavirus, and influenza A virus. PLOS Pathogens 7, (2011).
Kettleborough C A, Saldanha J, Heath V J, Morrison C J, Bendig M M. “Humanization of a mouse monoclonal antibody by CDR-grafting: the importance of framework residues on loop conformation”. Protein Eng. 1991 October;4(7):773-83.
Kohler G, Milstein C. Continuous cultures of fused cells secreting antibody of predefined specificity. Nature 1975; 256:495-497.
Yánez D C, Ross S, Crompton T. The IFITM protein family in adaptive immunity. Immunology. 2020;159(4):365-372.

1. An antibody or a fragment thereof which binds the N-terminal domain of IFITM-2 or IFITM-3 obtained by immunizing a mouse with a peptide consisting of an amino acid sequence as in SEQ ID NO: 38 or SEQ ID NO: 39.

2. The antibody or a fragment thereof according to claim 1, comprising a combination of a heavy chain or a heavy chain variable domain thereof and a light chain or a light chain variable domain thereof selected from the group of combinations consisting of:
a) a heavy chain amino acid sequence as in SEQ ID NO. 3 or a variable domain thereof or an amino acid sequence having at least 80% identity with the amino acid sequence in SEQ ID N. 3 or a variable domain thereof, and a light chain amino acid sequence as in SEQ ID NO. 4 or a variable domain thereof or an amino acid sequence having at least 80% identity with the amino acid sequence in SEQ ID N. 4 or a variable domain thereof;
b) a heavy chain amino acid sequence as in SEQ ID NO. 13 or a variable domain thereof or an amino acid sequence having at least 80% identity with the amino acid sequence in SEQ ID N: 13 or a variable domain thereof, and a light chain amino acid sequence as in SEQ ID NO: 14 or a variable domain thereof or an amino acid sequence having at least 80% identity with the amino acid sequence in SEQ ID NO. 14 or a variable domain thereof;
c) a heavy chain amino acid sequence as in SEQ ID NO: 23 or a variable domain thereof or an amino acid sequence having at least 80% identity with the amino acid sequence as in SEQ ID NO. 23 or a variable domain thereof, and a light chain amino acid sequence as in SEQ ID NO: 24 or a variable domain thereof or an amino acid sequence having at least 80% identity with the amino acid sequence as in SEQ ID. NO. 24 or a variable domain thereof; and
d) a heavy chain amino acid sequence as in SEQ ID NO: 33 or a variable domain thereof or an amino acid sequence having at least 80% identity with the amino acid sequence as in SEQ ID NO. 33 or a variable domain thereof, and a light chain amino acid sequence as in SEQ ID NO: 34 or a variable domain thereof or an amino acid sequence having at least 80% identity with the amino acid sequence as in SEQ ID NO. 34 or a variable domain thereof.

3. The antibody or a fragment thereof according to claim 2, comprising a combination of a heavy chain or a heavy chain variable domain and a light chain or a light chain variable domain thereof selected from the group of combinations consisting of:
a) a heavy chain amino acid sequence as in SEQ ID NO. 3 or a variable domain thereof or an amino acid sequence having at least 90% identity, with the amino acid sequence in SEQ ID N. 3 or a variable domain thereof, and a light chain amino acid sequence as in SEQ ID NO. 4 or a variable domain thereof or an amino acid sequence having at least 90% identity, with the amino acid sequence in SEQ ID N. 4 or a variable domain thereof;
b) a heavy chain amino acid sequence as in SEQ ID NO. 13 or a variable domain thereof or an amino acid sequence having at least 90% identity, with the amino acid sequence in SEQ ID N: 13 or a variable domain thereof, and a light chain amino acid sequence as in SEQ ID NO: 14 or a variable domain thereof or an amino acid sequence having at least 90% identity, with the amino acid sequence in SEQ ID NO. 14 or a variable domain thereof;
c) a heavy chain amino acid sequence as in SEQ ID NO: 23 or a variable domain thereof or an amino acid sequence having at least 90% identity, with the amino acid sequence as in SEQ ID NO. 23 or a variable domain thereof, and a light chain amino acid sequence as in SEQ ID NO: 24 or a variable domain thereof or an amino acid sequence having at least 90% identity, with the amino acid sequence as in SEQ ID. NO. 24 or a variable domain thereof; and
d) a heavy chain amino acid sequence as in SEQ ID NO: 33 or a variable domain thereof or an amino acid sequence having at least 90% identity, with the amino acid sequence as in SEQ ID NO. 33 or a variable domain thereof, and a light chain amino acid sequence as in SEQ ID NO: 34 or a variable domain thereof or an amino acid sequence having at least 90% identity, with the amino acid sequence as in SEQ ID NO. 34 or a variable domain thereof.

4. The antibody or a fragment thereof according to claim 2, wherein in the combination of a heavy chain amino acid sequence or a heavy chain variable domain thereof and of a light chain amino acid sequence or a light chain variable domain thereof at point a) the heavy chain amino acid sequence or at least the variable domain thereof comprises CDRs regions having an amino acid composition: H-CDR1 comprises amino acids SYAMS (SEQ ID N. 5), H-CDR2 comprises amino acids TITSGGSYTYYTDSVKG (SEQ ID N. 6), H-CDR3 comprises amino acids LMITTGWYFDV (SEQ ID N. 7) and the light chain amino acid sequence or the variable domain thereof comprises CDRs regions having an amino acid composition: L-CDR1 comprises amino acids RSSQSIVHSNGNTYLE (SEQ ID N. 8), L-CDR2 comprises amino acids KVSNRFS (SEQ ID N. 9) and L-CDR3 comprises amino acids FQGSHIPFT (SEQ ID N. 10).

5. The antibody or a fragment thereof according to claim 2, wherein in the combination of a heavy chain amino acid sequence or a heavy chain variable domain thereof and of a light chain amino acid sequence or a light chain variable domain thereof at point b) the heavy chain amino acid sequence or the variable domain thereof comprises CDRs regions having an amino acid composition: H-CDR1 comprises amino acids NYWMN (SEQ ID N. 15), H-CDR2 comprises amino acids EIRLKSNNYATHYAESVKG (SEQ ID N. 16), H-CDR3 comprises amino acids TLDY (SEQ ID N. 17) and the light chain amino acid sequence or the variable domain thereof comprises CDRs regions having an amino acid composition: L-CDR1 comprises amino acids KSSQSLLYSTNQKNYLA (SEQ ID N. 18), L-CDR2 comprises amino acids WASTRES (SEQ ID N. 19) and L-CDR3 comprises amino acids LQYYSYPYT (SEQ ID N. 20).

6. The antibody or a fragment thereof according to claim 2, wherein in the combination of a heavy chain amino acid sequence or a heavy chain variable domain thereof and a light chain amino acid sequence or a light chain variable domain thereof at point c) the heavy chain amino acid sequence or the variable domain thereof comprises CDRs regions having an amino acid composition: H-CDR 1 comprises amino acids DYYIH (SEQ ID N. 25), H-CDR2 comprises amino acids WINPENGNTMYDPKFQG (SEQ ID N. 26), H-CDR3 comprises amino acids DVYW (SEQ ID N. 27) and the light chain amino acid sequence or the variable domain thereof comprises CDRs regions having an amino acid composition: L-CDR1comprises amino acids RSSQSLVHSNGNTYLH (SEQ ID N. 28), L-CDR2 comprises amino acids KVSNRFS (SEQ ID N. 29) and L-CDR3 comprises amino acids SQSTHVPLT (SEQ ID N. 30).

7. The antibody or a fragment thereof according to claim 2, wherein in the combination of a heavy chain amino acid sequence or a heavy chain variable domain thereof and of a light chain amino acid sequence or a light chain variable domain thereof at point d) the heavy chain amino acid sequence or the variable domain thereof comprises CDRs regions having an amino acid composition: H-CDR1 comprises amino acids GYYMH (SEQ ID N. 35), H-CDR2comprises amino acids HINPYNGATSYNQNFKD (SEQ ID N. 36), H-CDR3 comprises amino acids DTYW (SEQ ID N. 37) and the light chain amino acid sequence or the variable domain thereof comprises CDRs regions having an amino acid composition: L-CDR1 comprises amino acids RSSQSLVHSNGNTYLH (SEQ ID N. 28), L-CDR2 comprises amino acids KVSNRFS (SEQ ID N. 29) and L-CDR3 comprises amino acids SQSTHVPLT (SEQ ID N. 30).

8. The antibody or a fragment thereof according to claim 1, wherein it is taken from the group consisting of a polyclonal or a monoclonal antibody, an antibody of natural or of synthetic origin, an antibody of mammalian origin, and a humanized antibody.

9. The antibody or a fragment thereof according to claim 1, characterized in that said antibody is a F(ab) fragment, a F(ab′) fragment, a F(ab′)₂ fragment, a Fv fragment, a diabody, a ScFv, a small modular immunopharmaceutical (SMIP), an affibody, an avimer, a nanobody, a domain antibody and/or single chains.

10. A pharmaceutical composition comprising at least one antibody or a fragment thereof according to claim 1 and at least one pharmaceutically acceptable excipient or carrier.

11. The pharmaceutical composition according to claim 10, wherein said composition is formulated in a form suitable for oral administration or in a form suitable for parenteral or topical administration.

12. The pharmaceutical composition according to claim 10 further comprising an active principle, selected from the group consisting of monoclonal antibodies, antiviral drugs as entry blockers, nucleoside/nucleoside analogues and non-nucleoside analogues, IFNs or protease inhibitors, anthelmintic drugs and antimalarial drugs.

13. A method for the treatment of a viral infection caused by a virus classified in the family of Adenoviridae, Anelloviridae, Arenaviridae, Astroviridae, Bornaviridaca, Bunyaviridae, Caliciviridae, Coronaviridae, Filoviridaca, Flaviviridae, Hepadnaviridae, Hepeviridae, Herpesviridae, Orthomyxoviridae, Papillomaviridac, Paramyxoviridaca, Parvoviridae, Picobirnaviridac, Picobirna, Picornaviridae, Pneumoviridae, Polyomaviridae, Poxviridac, Reoviridae, Retroviridae, Rhabdoviridaca, Togaviridae or Deltac comprising administering the antibody or a fragment thereof according to claim 1.

14. The antibody or a fragment thereof according to claim 3, wherein in the combination of a heavy chain amino acid sequence or a heavy chain variable domain thereof and a light chain amino acid sequence or a light chain variable domain thereof at point a) the heavy chain amino acid sequence or the variable domain thereof comprises CDRs regions having an amino acid composition: H-CDR1 comprises amino acids SYAMS (SEQ ID N. 5), H-CDR2 comprises amino acids TITSGGSYTYYTDSVKG (SEQ ID N. 6), H-CDR3 comprises amino acids LMITTGWYFDV (SEQ ID N. 7) and the light chain amino acid sequence or the variable domain thereof comprises CDRs regions having an amino acid composition: L-CDR1 comprises amino acids RSSQSIVHSNGNTYLE (SEQ ID N. 8), L-CDR2 comprises amino acids KVSNRFS (SEQ ID N. 9) and L-CDR3 comprises amino acids FQGSHIPFT (SEQ ID N. 10).

15. The antibody or a fragment thereof according to claim 3, wherein in the combination of a heavy chain amino acid sequence or a heavy chain variable domain thereof and a light chain amino acid sequence or a light chain variable domain thereof at point b) the heavy chain amino acid sequence or the variable domain thereof comprises CDRs regions having an amino acid composition: H-CDR1 comprises amino acids NYWMN (SEQ ID N. 15), H-CDR2 comprises amino acids EIRLKSNNYATHYAESVKG (SEQ ID N. 16), H-CDR3 comprises amino acids TLDY (SEQ ID N. 17) and the light chain amino acid sequence or the variable domain thereof comprises CDRs regions having an amino acid composition: L-CDR1 comprises amino acids KSSQSLLYSTNQKNYLA (SEQ ID N. 18), L-CDR2 comprises amino acids WASTRES (SEQ ID N. 19) and L-CDR3 comprises amino acids LQYYSYPYT (SEQ ID N. 20).

16. The antibody or a fragment thereof according to claim 3, wherein in the combination of a heavy chain amino acid sequence or a heavy chain variable domain thereof and a light chain amino acid sequence or a light chain variable domain thereof at point c) the heavy chain amino acid sequence or the variable domain thereof comprises CDRs regions having an amino acid composition: H-CDR1 comprises amino acids DYYIH (SEQ ID N. 25), H-CDR2 comprises amino acids WINPENGNTMYDPKFQG (SEQ ID N. 26), H-CDR3 comprises amino acids DVYW (SEQ ID N. 27) and the light chain amino acid sequence or the variable domain thereof comprises CDRs regions having an amino acid composition: L-CDR1 comprises amino acids RSSQSLVHSNGNTYLH (SEQ ID N. 28), L-CDR2 comprises amino acids KVSNRFS (SEQ ID N. 29) and L-CDR3 comprises amino acids SQSTHVPLT (SEQ ID N. 30).

17. The antibody or a fragment thereof according to claim 3, wherein in the combination of a heavy chain amino acid sequence or a heavy chain variable domain thereof and a light chain amino acid sequence or a light chain variable domain thereof at point d) the heavy chain amino acid sequence or the variable domain thereof comprises CDRs regions having an amino acid composition: H-CDR1 comprises amino acids GYYMH (SEQ ID N. 35), H-CDR2 comprises amino acids HINPYNGATSYNQNFKD (SEQ ID N. 36), H-CDR3 comprises amino acids DTYW (SEQ ID N. 37) and the light chain amino acid sequence or the variable domain thereof comprises CDRs regions having an amino acid composition: L-CDR1 comprises amino acids RSSQSLVHSNGNTYLH (SEQ ID N. 28), L-CDR2 comprises amino acids KVSNRFS (SEQ ID N. 29) and L-CDR3 comprises amino acids SQSTHVPLT (SEQ ID N. 30).

18. A method for the treatment of a viral infection caused by a virus classified in the family of Adenoviridae, Anelloviridae, Arenaviridae, Astroviridae, Bornaviridaea, Bunyaviridae, Caliciviridae, Coronaviridae, Filoviridaea, Flaviviridae, Hepadnaviridae, Hepeviridae, Herpesviridae, Orthomyxoviridae, Papillomaviridae, Paramyxoviridaea, Parvoviridae, Picobirnaviridae, Picobirna, Picornaviridae, Pneumoviridae, Polyomaviridae, Poxviridae, Reoviridae, Retroviridae, Rhabdoviridaea. Togaviridae or Deltae comprising administering the pharmaceutical composition of claim 10.

19. The antibody or a fragment thereof according to claim 2, comprising a combination of a heavy chain or a heavy chain variable domain and a light chain or a light chain variable domain thereof selected from the group of combinations consisting of:
a) a heavy chain amino acid sequence as in SEQ ID NO. 3 or variable domain thereof or an amino acid sequence having at least 95% identity with the amino acid sequence in SEQ ID N. 3 or a variable domain thereof, and a light chain amino acid sequence as in SEQ ID NO. 4 or a variable domain thereof or an amino acid sequence having at least 95% identity with the amino acid sequence in SEQ ID N. 4 or a variable domain thereof;
b) a heavy chain amino acid sequence as in SEQ ID NO. 13 or a variable domain thereof or an amino acid sequence having at least 95% identity with the amino acid sequence in SEQ ID N: 13 or a variable domain thereof, and a light chain amino acid sequence as in SEQ ID NO: 14 or a variable domain thereof or an amino acid sequence having at least 95% identity with the amino acid sequence in SEQ ID NO. 14 or a variable domain thereof;
c) a heavy chain amino acid sequence as in SEQ ID NO: 23 or a variable domain thereof or an amino acid sequence having at least 95% identity with the amino acid sequence as in SEQ ID NO. 23 or a variable domain thereof, and a light chain amino acid sequence as in SEQ ID NO: 24 or a variable domain thereof or an amino acid sequence having at least 95% identity with the amino acid sequence as in SEQ ID. NO. 24 or a variable domain thereof; and
d) a heavy chain amino acid sequence as in SEQ ID NO: 33 or a variable domain thereof or an amino acid sequence having at least 95% identity with the amino acid sequence as in SEQ ID NO. 33 or a variable domain thereof, and a light chain amino acid sequence as in SEQ ID NO: 34 or a variable domain thereof or an amino acid sequence having at least 95% identity with the amino acid sequence as in SEQ ID NO. 34 or a variable domain thereof.
